SEQUENCE LISTING (44 sequences)

SEQ ID NO: 1 - DNA, 7698 bp, Artificial Sequence
  Description of Artificial Sequence Synthetic polynucleotide
gaattcaccggtgccgccaccatgaagtgggtgaccttcatcagcctgctgttcctgttcagcagcgcctacagcgaggtgaagctggaggagagcggccccggcctggtggcccccagccagagcctgagcatcacctgcaccgtgagcggcttcagcctgacccgctacggcgtgagctgggtgcgccagccccccggcaagggcctggagtggctgggcgtgatctggggcgacggcaccaccaactaccacagcgccctgatcagccgcctgagcatcagcaaggacaacagcaagagccaggtgttcctgaagctgaacagcctgcagaccgacgacaccgccacctactactgcgcccgcacccgcaccggcggctactacgactacgtgatggactactggggccagggcaccagcgtgaccgtgagcagcggcggcggcggcagcggcggcggcggcagcggcggcggcggcagcggcggcggcggcagcgacatcgtgctgacccagaccaccgccagcctggccgtgcccctgggccagggcgccaccatcagctgccgcgccagcgagagcgtggagtactacgtgaccagcctgatgcagtggttccagcagaagcccggccagccccccaagctgctgatcagcgccgccagcaacgtggagagcggcgtgcccgcccgcttcagcggcagcggcagcggcaccgacttcaccctgaacatccaccccgtggaggaggacgacatcgccatgtacttctgccagcagagccgcaaggtgccctggaccttcggcggcggcaccaagctggagatcaagaccggtggatcctggagccacccccagttcgagaaggagcccaagagctgcgacaagacccacacctgccccttctgggtgctggtggtggtgggcggcgtgctggcctgctacagcctgctggtgaccgtggccttcatcatcttctgggtgagaagcaagcgcagccgcctgctgcacagcgactacatgaacatgaccccccgccgccccggccccacccgcaagcactaccagccctacgcccccccccgcgacttcgccgcctaccgcagcggcggcggcggcagcctgcgcgtgaagttcagccgcagcgccgacgcccccgcctaccagcagggccagaaccagctgtacaacgagctgaacctgggccgccgcgaggagtacgacgtgctggacaagcgccgcggcggcggcggcggcagccgcgaccagcgcctgccccccgacgcccacaagccccccggcggcggcagcttccgcacccccatccaggaggagcaggccgacgcccacagcaccctggccaagatcggatccagcgctggcagcggcgccacgaacttctctctgttaaagcaagcaggagacgtggaagaaaaccccggtcctatgaagtgggtgaccttcatcagcctgctgttcctgttcagcagcgcctacagcgacatcgtgctgacccagaccaccgccagcctggccgtgcccctgggccagggcgccaccatcagctgccgcgccagcgagagcgtggagtactacgtgaccagcctgatgcagtggttccagcagaagcccggccagccccccaagctgctgatcagcgccgccagcaacgtggagagcggcgtgcccgcccgcttcagcggcagcggcagcggcaccgacttcaccctgaacatccaccccgtggaggaggacgacatcgccatgtacttctgccagcagagccgcaaggtgccctggaccttcggcggcggcaccaagctggagatcaagggcggcggcggcagcggcggcggcggcagcggcggcggcggcagcggcggcggcggcagcgaggtgaagctggaggagagcggccccggcctggtggcccccagccagagcctgagcatcacctgcaccgtgagcggcttcagcctgacccgctacggcgtgagctgggtgcgccagccccccggcaagggcctggagtggctgggcgtgatctggggcgacggcaccaccaactaccacagcgccctgatcagccgcctgagcatcagcaaggacaacagcaagagccaggtgttcctgaagctgaacagcctgcagaccgacgacaccgccacctactactgcgcccgcacccgcaccggcggctactacgactacgtgatggactactggggccagggcaccagcgtgaccgtgagcagcggcggcggcggcagcggcggcggcggcagcggcggcggcggcagcggcggcggcggcagccaggtgcagctggtgcagagcggcggcggcgtggtgcagcccggccgcagcctgcgcctgagctgcaagagcagcggctacaccttcacccgctacaccatgcactgggtgcgccaggcccccggcaagggcctggagtggatcggctacatcaaccccagccgcggctacaccaactacaaccagaaggtgaaggaccgcttcaccatcagccgcgacaacagcaagaacaccgccttcctgcagatggacagcctgcgccccgaggacaccggcgtgtacttctgcgcccgctactacgacgaccactactgcctggactactggggccagggcacccccgtgaccgtgagcagcagcagcggcggcggcggcagcggcggcggcggcagcggcggcggcggcagcggcggcggcggcagcgacatccagatgacccagagccccagcagcctgagcgccagcgtgggcgaccgcgtgaccatcacctgccgcgccagcagcagcgtgagctacatgaactggtaccagcagacccccggcaaggcccccaagagatggatctacgacaccagcaaggtggccagcggcgtgcccagccgcttcagcggcagcggcagcggcaccgactacaccttcaccatcagcagcctgcagcccgaggacatcgccacctactactgccagcagtggagcagcaaccccctgaccttcggccagggcaccaagctgcagatcaccggcagcggcggcaagcccatccccaaccccctgctgggcctggacagcaccagcgctgcatgcggcagcggcgccacgaacttctctctgttaaagcaagcaggagacgtggaagaaaaccccggtcctatgaagtgggtgaccttcatcagcctgctgttcctgttcagcagcgcctacagcttccccgtgagcagcaaggagaagaacaccaagaccgtgcaggactacctggagaagttctaccagctgcccagcaaccagtaccagagcacccgcaagaacggcaccaacgtgatcgtggagaagctgaaggagatgcagagattcttcggcctgaacgtgaccggcaagcccaacgaggagaccctggacatgatgaagaagccccgctgcggcgtgcccgacagcggcggcttcatgctgacccccggcaaccccaagtgggagcgcaccaacctgacctaccgcatccgcaactacaccccccagctgagcgaggccgaggtggagcgcgccatcaaggacgccttcgagctgtggagcgtggccagccccctgatcttcacccgcatcagccagggcgaggccgacatcaacatcgccttctaccagcgcgaccacggcgacaacagccccttcgacggccccaacggcatcctggcccacgccttccagcccggccagggcatcggcggcgacgcccacttcgacgccgaggagacctggaccaacaccagcgccaactacaacctgttcctggtggccgcccacgagttcggccacagcctgggcctggcccacagcagcgaccccggcgccctgatgtaccccaactacgccttccgcgagaccagcaactacagcctgccccaggacgacatcgacggcatccaggccatctacggcctgagcagcaaccccatccagcccaccggccccagcacccccaagccctgcgaccccagcctgaccttcgacgccatcaccaccctgcgcggcgagatcctgttcttcaaggaccgctacttctggcgccgccacccccagctgcagcgcgtggagatgaacttcatcagcctgttctggcccagcctgcccaccggcatccaggccgcctacgaggacttcgaccgcgacctgatcttcctgttcaagggcaaccagtactgggccctgagcggctacgacatcctgcagggctaccccaaggacatcagcaactacggcttccccagcagcgtgcaggccatcgacgccgccgtgttctaccgcagcaagacctacttcttcgtgaacgaccagttctggcgctacgacaaccagcgccagttcatggagcccggctaccccaagagcatcagcggcgccttccccggcatcgagagcaaggtggacgccgtgttccagcaggagcacttcttccacgtgttcagcggcccccgctactacgccttcgacctgatcgcccagcgcgtgacccgcgtggcccgcggcaacaagtggctgaactgccgctacggcgcatgctgcgcaggcagcggcgccacgaacttctctctgttaaagcaagcaggagacgtggaagaaaaccccggtcctatgaagtgggtgaccttcatcagcctgctgttcctgttcagcagcgcctacagcagcgactgcgacatcgagggcaaggacggcaagcagtacgagagcgtgctgatggtgagcatcgaccagctgctggacagcatgaaggagatcggcagcaactgcctgaacaacgagttcaacttcttcaagcgccacatctgcgacgccaacaaggagggcatgttcctgttccgcgccgcccgcaagctgcgccagttcctgaagatgaacagcaccggcgacttcgacctgcacctgctgaaggtgagcgagggcaccaccatcctgctgaactgcaccggccaggtgaagggccgcaagcccgccgccctgggcgaggcccagcccaccaagagcctggaggagaacaagagcctgaaggagcagaagaagctgaacgacctgtgcttcctgaagcgcctgctgcaggagatcaagacctgctggaacaagatcctgatgggcaccaaggagcacggcagcggcgccacgaacttctctctgttaaagcaagcaggagacgtggaagaaaaccccggtcctatgaagtgggtgaccttcatcagcctgctgttcctgttcagcagcgcctacagccagatcccccaggccccctggcccgtggtgtgggccgtgctgcagctgggctggcgccccggctggttcctggacagccccgaccgcccctggaacccccccaccttcagccccgccctgctggtggtgaccgagggcgacaacgccaccttcacctgcagcttcagcaacaccagcgagagcttcgtgctgaactggtaccgcatgagccccagcaaccagaccgacaagctggccgccttccccgaggaccgcagccagcccggccaggactgccgcttccgcgtgacccagctgcccaacggccgcgacttccacatgagcgtggtgcgcgcccgccgcaacgacagcggcacctacctgtgcggcgccatcagcctggcccccaaggcccagatcaaggagagcctgcgcgccgagctgcgcgtgaccgagcgccgcgccgaggtgcccaccgcccaccccagccccagcccccgccccgccggccagttccagagcttcttcctggccctgaccagcaccgccctgctgttcctgctgttcttcctgaccctgcgcttcagcgtggtgaagcgcggccgcaagaagctgctgtacatcttcaagcagcccttcatgcgccccgtgcagaccacccaggaggaggacggctgcagctgccgcttccccgaggaggaggagggcggctgcgagctgggcagcggcgccacgaacttctctctgttaaagcaagcaggagacgtggaagaaaaccccggtcctatggtgctggaggtgagcgaccaccaggtgctgaacgacgccgaggtggccgccctgctggagaacttcagcagcagctacgactacggcgagaacgagagcgacagctgctgcaccagccccccctgcccccaggacttcagcctgaacttcgaccgcgccttcctgcccgccctgtacagcctgctgttcctgctgggcctgctgggcaacggcgccgtggccgccgtgctgctgagccgccgcaccgccctgagcagcaccgacaccttcctgctgcacctggccgtggccgacaccctgctggtgctgaccctgcccctgtgggccgtggacgccgccgtgcagtgggtgttcggcagcggcctgtgcaaggtggccggcgccctgttcaacatcaacttctacgccggcgccctgctgctggcctgcatcagcttcgaccgctacctgaacatcgtgcacgccacccagctgtaccgccgcggcccccccgcccgcgtgaccctgacctgcctggccgtgtggggcctgtgcctgctgttcgccctgcccgacttcatcttcctgagcgcccaccacgacgagcgcctgaacgccacccactgccagtacaacttcccccaggtgggccgcaccgccctgcgcgtgctgcagctggtggccggcttcctgctgcccctgctggtgatggcctactgctacgcccacatcctggccgtgctgctggtgagccgcggccagcgccgcctgcgcgccatgcgcctggtggtggtggtggtggtggccttcgccctgtgctggaccccctaccacctggtggtgctggtggacatcctgatggacctgggcgccctggcccgcaactgcggccgcgagagccgcgtggacgtggccaagagcgtgaccagcggcctgggctacatgcactgctgcctgaaccccctgctgtacgccttcgtgggcgtgaagttccgcgagcgcatgtggatgctgctgctgcgcctgggctgccccaaccagcgcggcctgcagcgccagcccagcagcagccgccgcgacagcagctggagcgagaccagcgaggccagctacagcggcctgggcagcggcgccacgaacttctctctgttaaagcaagcaggagacgtggaagaaaaccccggtcctatgaagtgggtgaccttcatcagcctgctgttcctgttcagcagcgcctacagccagggccaggaccgccacatgatcagaatgcgccagctgatcgacatcgtggaccagctgaagaactacgtgaacgacctggtgcccgagttcctgcccgcccccgaggacgtggagaccaactgcgagtggagcgccttcagctgcttccagaaggcccagctgaagagcgccaacaccggcaacaacgagcgcatcatcaacgtgagcatcaagaagctgaagcgcaagccccccagcaccaacgccggccgccgccagaagcaccgcctgacctgccccagctgcgacagctacgagaagaagccccccaaggagttcctggagagattcaagagcctgctgcagaagatgatccaccagcacctgagcagccgcacccacggcagcgaggacagctgcgcatgatgactcgag

SEQ ID NO: 2 - DNA, 54 bp, Artificial Sequence
  Description of Artificial Sequence Synthetic oligonucleotide
atgaagtgggtgaccttcatcagcctgctgttcctgttcagcagcgcctacagc

SEQ ID NO: 3 - protein, 18 aa, Artificial Sequence
  Description of Artificial Sequence Synthetic peptide
MKWVTFISLLFLFSSAYS

SEQ ID NO: 4 - DNA, 1305 bp, Artificial Sequence
  Description of Artificial Sequence Synthetic polynucleotide
gaggtgaagctggaggagagcggccccggcctggtggcccccagccagagcctgagcatcacctgcaccgtgagcggcttcagcctgacccgctacggcgtgagctgggtgcgccagccccccggcaagggcctggagtggctgggcgtgatctggggcgacggcaccaccaactaccacagcgccctgatcagccgcctgagcatcagcaaggacaacagcaagagccaggtgttcctgaagctgaacagcctgcagaccgacgacaccgccacctactactgcgcccgcacccgcaccggcggctactacgactacgtgatggactactggggccagggcaccagcgtgaccgtgagcagcggcggcggcggcagcggcggcggcggcagcggcggcggcggcagcggcggcggcggcagcgacatcgtgctgacccagaccaccgccagcctggccgtgcccctgggccagggcgccaccatcagctgccgcgccagcgagagcgtggagtactacgtgaccagcctgatgcagtggttccagcagaagcccggccagccccccaagctgctgatcagcgccgccagcaacgtggagagcggcgtgcccgcccgcttcagcggcagcggcagcggcaccgacttcaccctgaacatccaccccgtggaggaggacgacatcgccatgtacttctgccagcagagccgcaaggtgccctggaccttcggcggcggcaccaagctggagatcaagaccggtggatcctggagccacccccagttcgagaaggagcccaagagctgcgacaagacccacacctgccccttctgggtgctggtggtggtgggcggcgtgctggcctgctacagcctgctggtgaccgtggccttcatcatcttctgggtgagaagcaagcgcagccgcctgctgcacagcgactacatgaacatgaccccccgccgccccggccccacccgcaagcactaccagccctacgcccccccccgcgacttcgccgcctaccgcagcggcggcggcggcagcctgcgcgtgaagttcagccgcagcgccgacgcccccgcctaccagcagggccagaaccagctgtacaacgagctgaacctgggccgccgcgaggagtacgacgtgctggacaagcgccgcggcggcggcggcggcagccgcgaccagcgcctgccccccgacgcccacaagccccccggcggcggcagcttccgcacccccatccaggaggagcaggccgacgcccacagcaccctggccaagatcggatccagcgct

SEQ ID NO: 5 - protein, 435 aa, Artificial Sequence
  Description of Artificial Sequence Synthetic polypeptide
EVKLEESGPGLVAPSQSLSITCTVSGFSLTRYGVSWVRQPPGKGLEWLGVIWGDGTTNYHSALISRLSISKDNSKSQVFLKLNSLQTDDTATYYCARTRTGGYYDYVMDYWGQGTSVTVSSGGGGSGGGGSGGGGSGGGGSDIVLTQTTASLAVPLGQGATISCRASESVEYYVTSLMQWFQQKPGQPPKLLISAASNVESGVPARFSGSGSGTDFTLNIHPVEEDDIAMYFCQQSRKVPWTFGGGTKLEIKTGGSWSHPQFEKEPKSCDKTHTCPFWVLVVVGGVLACYSLLVTVAFIIFWVRSKRSRLLHSDYMNMTPRRPGPTRKHYQPYAPPRDFAAYRSGGGGSLRVKFSRSADAPAYQQGQNQLYNELNLGRREEYDVLDKRRGGGGGSRDQRLPPDAHKPPGGGSFRTPIQEEQADAHSTLAKIGSSA

SEQ ID NO: 6 - DNA, 66 bp, Artificial Sequence
  Description of Artificial Sequence Synthetic oligonucleotide
ggcagcggcgccacgaacttctctctgttaaagcaagcaggagacgtggaagaaaaccccggtcct

SEQ ID NO: 7 - protein, 22 aa, Artificial Sequence
  Description of Artificial Sequence Synthetic peptide
GSGATNFSLLKQAGDVEENPGP

SEQ ID NO: 8 - DNA, 54 bp, Artificial Sequence; identical to SEQ ID NO: 2
  Description of Artificial Sequence Synthetic oligonucleotide

SEQ ID NO: 9 - protein, 18 aa, Artificial Sequence
  Description of Artificial Sequence Synthetic peptide
MKWVTFISLLFLFSSAYS

SEQ ID NO: 10 - DNA, 1557 bp, Artificial Sequence
  Description of Artificial Sequence Synthetic polynucleotide
gacatcgtgctgacccagaccaccgccagcctggccgtgcccctgggccagggcgccaccatcagctgccgcgccagcgagagcgtggagtactacgtgaccagcctgatgcagtggttccagcagaagcccggccagccccccaagctgctgatcagcgccgccagcaacgtggagagcggcgtgcccgcccgcttcagcggcagcggcagcggcaccgacttcaccctgaacatccaccccgtggaggaggacgacatcgccatgtacttctgccagcagagccgcaaggtgccctggaccttcggcggcggcaccaagctggagatcaagggcggcggcggcagcggcggcggcggcagcggcggcggcggcagcggcggcggcggcagcgaggtgaagctggaggagagcggccccggcctggtggcccccagccagagcctgagcatcacctgcaccgtgagcggcttcagcctgacccgctacggcgtgagctgggtgcgccagccccccggcaagggcctggagtggctgggcgtgatctggggcgacggcaccaccaactaccacagcgccctgatcagccgcctgagcatcagcaaggacaacagcaagagccaggtgttcctgaagctgaacagcctgcagaccgacgacaccgccacctactactgcgcccgcacccgcaccggcggctactacgactacgtgatggactactggggccagggcaccagcgtgaccgtgagcagcggcggcggcggcagcggcggcggcggcagcggcggcggcggcagcggcggcggcggcagccaggtgcagctggtgcagagcggcggcggcgtggtgcagcccggccgcagcctgcgcctgagctgcaagagcagcggctacaccttcacccgctacaccatgcactgggtgcgccaggcccccggcaagggcctggagtggatcggctacatcaaccccagccgcggctacaccaactacaaccagaaggtgaaggaccgcttcaccatcagccgcgacaacagcaagaacaccgccttcctgcagatggacagcctgcgccccgaggacaccggcgtgtacttctgcgcccgctactacgacgaccactactgcctggactactggggccagggcacccccgtgaccgtgagcagcagcagcggcggcggcggcagcggcggcggcggcagcggcggcggcggcagcggcggcggcggcagcgacatccagatgacccagagccccagcagcctgagcgccagcgtgggcgaccgcgtgaccatcacctgccgcgccagcagcagcgtgagctacatgaactggtaccagcagacccccggcaaggcccccaagagatggatctacgacaccagcaaggtggccagcggcgtgcccagccgcttcagcggcagcggcagcggcaccgactacaccttcaccatcagcagcctgcagcccgaggacatcgccacctactactgccagcagtggagcagcaaccccctgaccttcggccagggcaccaagctgcagatcacc

SEQ ID NO: 11 - protein, 519 aa, Artificial Sequence
  Description of Artificial Sequence Synthetic polypeptide
DIVLTQTTASLAVPLGQGATISCRASESVEYYVTSLMQWFQQKPGQPPKLLISAASNVESGVPARFSGSGSGTDFTLNIHPVEEDDIAMYFCQQSRKVPWTFGGGTKLEIKGGGGSGGGGSGGGGSGGGGSEVKLEESGPGLVAPSQSLSITCTVSGFSLTRYGVSWVRQPPGKGLEWLGVIWGDGTTNYHSALISRLSISKDNSKSQVFLKLNSLQTDDTATYYCARTRTGGYYDYVMDYWGQGTSVTVSSGGGGSGGGGSGGGGSGGGGSQVQLVQSGGGVVQPGRSLRLSCKSSGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSSSGGGGSGGGGSGGGGSGGGGSDIQMTQSPSSLSASVGDRVTITCRASSSVSYMNWYQQTPGKAPKRWIYDTSKVASGVPSRFSGSGSGTDYTFTISSLQPEDIATYYCQQWSSNPLTFGQGTKLQIT

SEQ ID NO: 12 - DNA, 42 bp, Artificial Sequence
  Description of Artificial Sequence Synthetic oligonucleotide
ggcaagcccatccccaaccccctgctgggcctggacagcacc

SEQ ID NO: 13 - protein, 14 aa, Artificial Sequence
  Description of Artificial Sequence Synthetic peptide
GKPIPNPLLGLDST

SEQ ID NO: 14 - DNA, 66 bp, Artificial Sequence; identical to SEQ ID NO: 6
  Description of Artificial Sequence Synthetic oligonucleotide

SEQ ID NO: 15 - protein, 22 aa, Artificial Sequence
  Description of Artificial Sequence Synthetic peptide
GSGATNFSLLKQAGDVEENPGP

SEQ ID NO: 16 - DNA, 54 bp, Artificial Sequence; identical to SEQ ID NO: 2
  Description of Artificial Sequence Synthetic oligonucleotide

SEQ ID NO: 17 - protein, 18 aa, Artificial Sequence
  Description of Artificial Sequence Synthetic peptide
MKWVTFISLLFLFSSAYS

SEQ ID NO: 18 - DNA, 1341 bp, Artificial Sequence
  Description of Artificial Sequence Synthetic polynucleotide
ttccccgtgagcagcaaggagaagaacaccaagaccgtgcaggactacctggagaagttctaccagctgcccagcaaccagtaccagagcacccgcaagaacggcaccaacgtgatcgtggagaagctgaaggagatgcagagattcttcggcctgaacgtgaccggcaagcccaacgaggagaccctggacatgatgaagaagccccgctgcggcgtgcccgacagcggcggcttcatgctgacccccggcaaccccaagtgggagcgcaccaacctgacctaccgcatccgcaactacaccccccagctgagcgaggccgaggtggagcgcgccatcaaggacgccttcgagctgtggagcgtggccagccccctgatcttcacccgcatcagccagggcgaggccgacatcaacatcgccttctaccagcgcgaccacggcgacaacagccccttcgacggccccaacggcatcctggcccacgccttccagcccggccagggcatcggcggcgacgcccacttcgacgccgaggagacctggaccaacaccagcgccaactacaacctgttcctggtggccgcccacgagttcggccacagcctgggcctggcccacagcagcgaccccggcgccctgatgtaccccaactacgccttccgcgagaccagcaactacagcctgccccaggacgacatcgacggcatccaggccatctacggcctgagcagcaaccccatccagcccaccggccccagcacccccaagccctgcgaccccagcctgaccttcgacgccatcaccaccctgcgcggcgagatcctgttcttcaaggaccgctacttctggcgccgccacccccagctgcagcgcgtggagatgaacttcatcagcctgttctggcccagcctgcccaccggcatccaggccgcctacgaggacttcgaccgcgacctgatcttcctgttcaagggcaaccagtactgggccctgagcggctacgacatcctgcagggctaccccaaggacatcagcaactacggcttccccagcagcgtgcaggccatcgacgccgccgtgttctaccgcagcaagacctacttcttcgtgaacgaccagttctggcgctacgacaaccagcgccagttcatggagcccggctaccccaagagcatcagcggcgccttccccggcatcgagagcaaggtggacgccgtgttccagcaggagcacttcttccacgtgttcagcggcccccgctactacgccttcgacctgatcgcccagcgcgtgacccgcgtggcccgcggcaacaagtggctgaactgccgctacggc

SEQ ID NO: 19 - protein, 447 aa, Artificial Sequence
  Description of Artificial Sequence Synthetic polypeptide
FPVSSKEKNTKTVQDYLEKFYQLPSNQYQSTRKNGTNVIVEKLKEMQRFFGLNVTGKPNEETLDMMKKPRCGVPDSGGFMLTPGNPKWERTNLTYRIRNYTPQLSEAEVERAIKDAFELWSVASPLIFTRISQGEADINIAFYQRDHGDNSPFDGPNGILAHAFQPGQGIGGDAHFDAEETWTNTSANYNLFLVAAHEFGHSLGLAHSSDPGALMYPNYAFRETSNYSLPQDDIDGIQAIYGLSSNPIQPTGPSTPKPCDPSLTFDAITTLRGEILFFKDRYFWRRHPQLQRVEMNFISLFWPSLPTGIQAAYEDFDRDLIFLFKGNQYWALSGYDILQGYPKDISNYGFPSSVQAIDAAVFYRSKTYFFVNDQFWRYDNQRQFMEPGYPKSISGAFPGIESKVDAVFQQEHFFHVFSGPRYYAFDLIAQRVTRVARGNKWLNCRYG

SEQ ID NO: 20 - DNA, 66 bp, Artificial Sequence; identical to SEQ ID NO: 6
  Description of Artificial Sequence Synthetic oligonucleotide

SEQ ID NO: 21 - protein, 22 aa, Artificial Sequence
  Description of Artificial Sequence Synthetic peptide
GSGATNFSLLKQAGDVEENPGP

SEQ ID NO: 22 - DNA, 54 bp, Artificial Sequence; identical to SEQ ID NO: 2
  Description of Artificial Sequence Synthetic oligonucleotide

SEQ ID NO: 23 - protein, 18 aa, Artificial Sequence
  Description of Artificial Sequence Synthetic peptide
MKWVTFISLLFLFSSAYS

SEQ ID NO: 24 - DNA, 459 bp, Artificial Sequence
  Description of Artificial Sequence Synthetic polynucleotide
agcgactgcgacatcgagggcaaggacggcaagcagtacgagagcgtgctgatggtgagcatcgaccagctgctggacagcatgaaggagatcggcagcaactgcctgaacaacgagttcaacttcttcaagcgccacatctgcgacgccaacaaggagggcatgttcctgttccgcgccgcccgcaagctgcgccagttcctgaagatgaacagcaccggcgacttcgacctgcacctgctgaaggtgagcgagggcaccaccatcctgctgaactgcaccggccaggtgaagggccgcaagcccgccgccctgggcgaggcccagcccaccaagagcctggaggagaacaagagcctgaaggagcagaagaagctgaacgacctgtgcttcctgaagcgcctgctgcaggagatcaagacctgctggaacaagatcctgatgggcaccaaggagcac

SEQ ID NO: 25 - protein, 153 aa, Artificial Sequence
  Description of Artificial Sequence Synthetic polypeptide
SDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 26 - DNA, 66 bp, Artificial Sequence; identical to SEQ ID NO: 6
  Description of Artificial Sequence Synthetic oligonucleotide

SEQ ID NO: 27 - protein, 22 aa, Artificial Sequence
  Description of Artificial Sequence Synthetic peptide
GSGATNFSLLKQAGDVEENPGP

SEQ ID NO: 28 - DNA, 54 bp, Artificial Sequence; identical to SEQ ID NO: 2
  Description of Artificial Sequence Synthetic oligonucleotide

SEQ ID NO: 29 - protein, 18 aa, Artificial Sequence
  Description of Artificial Sequence Synthetic peptide
MKWVTFISLLFLFSSAYS

SEQ ID NO: 30 - DNA, 699 bp, Artificial Sequence
  Description of Artificial Sequence Synthetic polynucleotide
cagatcccccaggccccctggcccgtggtgtgggccgtgctgcagctgggctggcgccccggctggttcctggacagccccgaccgcccctggaacccccccaccttcagccccgccctgctggtggtgaccgagggcgacaacgccaccttcacctgcagcttcagcaacaccagcgagagcttcgtgctgaactggtaccgcatgagccccagcaaccagaccgacaagctggccgccttccccgaggaccgcagccagcccggccaggactgccgcttccgcgtgacccagctgcccaacggccgcgacttccacatgagcgtggtgcgcgcccgccgcaacgacagcggcacctacctgtgcggcgccatcagcctggcccccaaggcccagatcaaggagagcctgcgcgccgagctgcgcgtgaccgagcgccgcgccgaggtgcccaccgcccaccccagccccagcccccgccccgccggccagttccagagcttcttcctggccctgaccagcaccgccctgctgttcctgctgttcttcctgaccctgcgcttcagcgtggtgaagcgcggccgcaagaagctgctgtacatcttcaagcagcccttcatgcgccccgtgcagaccacccaggaggaggacggctgcagctgccgcttccccgaggaggaggagggcggctgcgagctg

SEQ ID NO: 31 - protein, 233 aa, Artificial Sequence
  Description of Artificial Sequence Synthetic polypeptide
QIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSFFLALTSTALLFLLFFLTLRFSVVKRGRKKLLYIFKQPFMRPVQTTQEEDGCSCRFPEEEEGGCEL

SEQ ID NO: 32 - DNA, 66 bp, Artificial Sequence; identical to SEQ ID NO: 6
  Description of Artificial Sequence Synthetic oligonucleotide

SEQ ID NO: 33 - protein, 22 aa, Artificial Sequence
  Description of Artificial Sequence Synthetic peptide
GSGATNFSLLKQAGDVEENPGP

SEQ ID NO: 34 - DNA, 1104 bp, Artificial Sequence
  Description of Artificial Sequence Synthetic polynucleotide
atggtgctggaggtgagcgaccaccaggtgctgaacgacgccgaggtggccgccctgctggagaacttcagcagcagctacgactacggcgagaacgagagcgacagctgctgcaccagccccccctgcccccaggacttcagcctgaacttcgaccgcgccttcctgcccgccctgtacagcctgctgttcctgctgggcctgctgggcaacggcgccgtggccgccgtgctgctgagccgccgcaccgccctgagcagcaccgacaccttcctgctgcacctggccgtggccgacaccctgctggtgctgaccctgcccctgtgggccgtggacgccgccgtgcagtgggtgttcggcagcggcctgtgcaaggtggccggcgccctgttcaacatcaacttctacgccggcgccctgctgctggcctgcatcagcttcgaccgctacctgaacatcgtgcacgccacccagctgtaccgccgcggcccccccgcccgcgtgaccctgacctgcctggccgtgtggggcctgtgcctgctgttcgccctgcccgacttcatcttcctgagcgcccaccacgacgagcgcctgaacgccacccactgccagtacaacttcccccaggtgggccgcaccgccctgcgcgtgctgcagctggtggccggcttcctgctgcccctgctggtgatggcctactgctacgcccacatcctggccgtgctgctggtgagccgcggccagcgccgcctgcgcgccatgcgcctggtggtggtggtggtggtggccttcgccctgtgctggaccccctaccacctggtggtgctggtggacatcctgatggacctgggcgccctggcccgcaactgcggccgcgagagccgcgtggacgtggccaagagcgtgaccagcggcctgggctacatgcactgctgcctgaaccccctgctgtacgccttcgtgggcgtgaagttccgcgagcgcatgtggatgctgctgctgcgcctgggctgccccaaccagcgcggcctgcagcgccagcccagcagcagccgccgcgacagcagctggagcgagaccagcgaggccagctacagcggcctg

SEQ ID NO: 35 - protein, 368 aa, Artificial Sequence
  Description of Artificial Sequence Synthetic polypeptide
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 36 - DNA, 66 bp, Artificial Sequence; identical to SEQ ID NO: 6
  Description of Artificial Sequence Synthetic oligonucleotide

SEQ ID NO: 37 - protein, 22 aa, Artificial Sequence
  Description of Artificial Sequence Synthetic peptide
GSGATNFSLLKQAGDVEENPGP

SEQ ID NO: 38 - DNA, 54 bp, Artificial Sequence; identical to SEQ ID NO: 2
  Description of Artificial Sequence Synthetic oligonucleotide

SEQ ID NO: 39 - protein, 18 aa, Artificial Sequence
  Description of Artificial Sequence Synthetic peptide
MKWVTFISLLFLFSSAYS

SEQ ID NO: 40 - DNA, 399 bp, Artificial Sequence
  Description of Artificial Sequence Synthetic polynucleotide
cagggccaggaccgccacatgatcagaatgcgccagctgatcgacatcgtggaccagctgaagaactacgtgaacgacctggtgcccgagttcctgcccgcccccgaggacgtggagaccaactgcgagtggagcgccttcagctgcttccagaaggcccagctgaagagcgccaacaccggcaacaacgagcgcatcatcaacgtgagcatcaagaagctgaagcgcaagccccccagcaccaacgccggccgccgccagaagcaccgcctgacctgccccagctgcgacagctacgagaagaagccccccaaggagttcctggagagattcaagagcctgctgcagaagatgatccaccagcacctgagcagccgcacccacggcagcgaggacagc

SEQ ID NO: 41 - protein, 133 aa, Artificial Sequence [sequence omitted]

SEQ ID NO: 42 - protein, 4 aa, Artificial Sequence
  Description of Artificial Sequence Synthetic peptide
GGGS

SEQ ID NO: 43 - protein, 16 aa, Artificial Sequence
  Description of Artificial Sequence Synthetic peptide
GGGSGGGSGGGSGGGS

SEQ ID NO: 44 - protein, 20 aa, Artificial Sequence
  Description of Artificial Sequence Synthetic peptide
GGGGSGGGGSGGGGSGGGGS

(19) United States
(12) Patent Application Publication
(10) Pub. No.: US 2022/0119478 A1
(43) Pub. Date: Apr. 21, 2022

(54) ADVANCED CHIMERIC ANTIGEN RECEPTOR VECTORS FOR TARGETING SOLID TUMORS

(71) Applicant: CAERUS THERAPEUTICS, CORP., Nokesville, VA (US)

(72) Inventors: Mark SPEAR, Nokesville, VA (US); Cohava GELBER, Nokesville, VA (US)

(73) Assignee: CAERUS THERAPEUTICS, CORP., Nokesville, VA (US)

(21) Appl. No.: 17/423,059

(22) PCT Filed: Jan. 15, 2020
(86) PCT No.: PCT/US20/13760

§ 371 Date: Jul. 14, 2021

Related U.S. Application Data
(60) Provisional application No. 62/792,511, filed on Jan. 15, 2019.

(51) Int. Cl. C07K 14/725; C12N 15/62; C12N 15/86; C12N 9/64; C07K 14/705; C07K 14/54; C07K 14/715

(52) U.S. Cl. CPC C07K 14/7051; C12N 15/62; C12N 15/86; C12N 9/6491; C07K 14/70521; C07K 2319/03; C07K 14/5418; C07K 14/7158; C12N 2320/30; C12N 2740/15043; C07K 14/70578

(57) ABSTRACT

Provided herein are constructs comprising a CAR nucleic acid sequence comprising a single chain variable fragment from monoclonal antibody VAC69 for use in stimulating an immune response against solid tumors. The CAR nucleic acid sequence is linked to nucleic acids encoding one or more cytokines, and one or more of a matrix metalloprotease, a PD1 fusion protein, a chemokine receptor, a dominant negative or nonfunctional immunosuppressive or toxic receptor, a FOXP3 inhibitory peptide P60, and a Bi-specific T Cell Engager (BiTE). The constructs can further include one or more of a signal peptide, a self-cleaving peptide, an epitope tag, an internal ribosome entry site (IRES), or a selectable marker.

### RELATED APPLICATION DATA

This application claims priority to U.S. Provisional Patent Application Ser. No. 62/792,511, filed Jan. 15, 2019, the contents of which are herein incorporated by reference in their entirety.

### BACKGROUND

Chimeric Antigen Receptors (CARs) are recombinant cell surface proteins that specifically target a known antigen, resulting in immune cell activation. They are protein fusions of either an antibody, single chain Fv antibody fragment (scFv), or a known receptor's ligand, which are fused with a transmembrane domain and an activating intracellular domain, such as CD3ζ intracellular domain. When expressed in B, T, NK, or NKT cells, the CAR bypasses canonical receptor-ligand activation and co-stimulation requirements, allowing for a universal method of antigen-dependent induction of effector functions. This process has been successfully utilized as CAR T cell therapies for targeting cancer; however, this technology has generated only marginal success in solid tumors.

### SUMMARY

Described herein is a cancer-specific single-chain Fv (scFv) from monoclonal antibody (mAb) VAC69, which is fused to a third generation CAR. This antibody has been previously shown to bind to an ovarian cancer (OVC) and multiple myeloma (MM) surface antigen, inducing peripheral blood mononuclear cell (PBMC)-mediated Antibody-Dependent Cellular Cytotoxicity (ADCC) [1]. Furthermore, this CAR is encoded on a lentivector along with several other effectors presumed or known to enhance immune function in solid tumors or other malignancies. Moreover, these additional factors mitigate the immunosuppressive effects of the tumor microenvironment, promote CAR-transduced cell survival and persistence in the host, and facilitate T, NK, or NKT cell infiltration into the tumor.

Provided herein is construct comprising a nucleic acid sequence encoding: a) a chimeric antigen receptor (CAR) comprising a single chain variable fragment from monoclonal antibody VAC69; b) one or more cytokines; and c) one or more of: i) a matrix metaloproteinase; ii) a PD1 fusion protein; iii) a chemokine receptor; iv) a dominant negative or nonfunctional immunosuppressive or toxic receptor. v) a FOXP3 inhibitory peptide P60; vi) a Bi-specific T Cell Engager (BiTE). The construct of can include a humanized single chain variable fragment from monoclonal antibody VAC69. The construct can include a third-generation CAR. The CAR can include the transmembrane and intracellular domains of CD28, the intracellular domain of CD3ζ, and the intracellular domain of OX40. The CAR can further include a glycine-glycine-glycine-serine (GGGS) linker. In some embodiments, the CAR comprises a nucleotide sequence at least 90% identical to SEQ ID NO. 4. In some embodiments, the CAR comprises the nucleotide sequence of SEQ ID NO. 4. The PD1 fusion protein can include a PD-1 extracellular domain (ECD) and a transmembrane domain (TMD) and an intracellular domain (ICD) of 4-1BB. In some embodiments, the PD1 fusion protein comprises a nucleotide sequence at least 90% identical to SEQ ID NO. 30. In some embodiments, PD1 fusion protein comprises the nucleotide sequence of SEQ ID NO. 30. The cytokine can be IL-7, IL-21, or both IL-7 and IL 21. In some embodiments, the IL-7 comprises a nucleotide sequence at least 90% identical to SEQ ID NO. 24. In some embodiments, the IL-7 comprises the nucleotide sequence of SED NO. 24. In some embodiments, the IL-21 comprises a nucleotide sequence at least 90% identical to SEQ ID NO. 40. In some embodiments, the IL-21 comprises the nucleotide sequence of SED NO. 40 The matrix metaloproteinase can be Pro-MMP-8. In some embodiments, the Pro-MMP-8 comprises a nucleotide sequence at least 90% identical to SEQ ID NO. 18. in some embodiments, the Pro-MMP-8 comprises the nucleotide sequence of SEQ ID NO. 18. The dominant negative or nonfunctional immunosuppressive or toxic receptor can be a dominant negative Fas-associated death domain protein (FADD). The Bi-specific T Cell Engager (BiTE) can be a fusion protein comprising a single chain variable fragment from monoclonal antibody VAC69 and a humanized single chain variable fragment monoclonal antibody OKT3. In some embodiments, the Bi-specific T Cell Engager (BiTE) comprises comprises a nucleotide sequence at least 90% identical to SEQ ID NO. 10. In some embodiments, the Bi-specific T Cell Engager (BiTE) comprises comprises the nucleotide sequence of SEQ ID NO. 10.

The construct can further comprise one or more of a signal peptide, a self cleaving peptide, an epitope tag, an internal ribosome entry site (IRES), or a selectable marker. The signal peptide can be a human serum albumin signal peptide. In some embodiments, human serum albumin signal peptide comprises a nucleotide sequence at least 90% identical to SEQ ID NO. 2, 8, 16, 22, 28, or 38. In some embodiments, the human serum albumin signal peptide comprises comprises the nucleotide sequence of SEQ ID NO. 2, 8, 16, 22, 28, or 38. The self-cleaving peptide can be a P2A peptide. In some embodiments, the P2A peptide comprises a nucleotide sequence at least 90% identical to SEQ ID NO. 6, 14, 20, 26, 32, or 36. In some embodiments, the P2A peptide comprises the nucleotide sequence of SEQ ID NO. 6, 14, 20, 26, 32, or 36. The epitope tag can be a V5 epitope tag. In some embodiments, the V5 epitope tag comprises a nucleotide sequence at least 90% identical to SEQ ID NO. 12. In some embodiments, the V5 epitope tag comprises the nucleotide sequence of SEQ ID NO. 12.

A construct can be configured to include a nucleic acid sequence encoding: a) a chimeric antigen receptor (CAR) comprising a single chain variable fragment from monoclonal antibody VAC69; b) a Bi-specific T Cell Engager (BiTE) comprising a fusion protein comprising a single chain variable fragment from monoclonal antibody VAC69 and a humanized single chain variable fragment monoclonal antibody OKT3; c) Pro-MMP-8; d) IL-7; e) a fusion protein comprising a PD1-extracellular domain and a 4-1BB transmembrane and intracellular domain; f) a CXCR3 chemokine receptor; and g) IL-21. The construct can further include one or more of a signal peptide, a self cleaving peptide, and an epitope tag. In some embodiments, the construct comprises a nucleotide sequence at least 90% identical to SEQ ID NO. 1. In some embodiments, the construct comprises the nucleotide sequence of SEQ ID NO. 1.

Also provided are vectors comprising any of the constructs disclosed herein. The the vector can be a lentiviral vector. In some embodiments, lentiviral vector can be a self inactivating lentiviral vector. Also provided are host cells comprising a vector containing any of the constructs disclosed herein.

Also provided are pharmaceutical compositions comprising any of the constructs disclosed herein.

Also provided are methods of treating a malignancy, the method comprising administering a pharmaceutical composition comprising any of the constructs disclosed herein. In some embodiments the constructs can be contained within a vector or a host cell. The malignancy can be a solid tumor.

### BRIEF DESCRIPTIONS OF DRAWINGS

This patent or application file contains at least one drawing executed in color. Copies of this patent or patent application publication with color drawing(s) will be provided by the Office upon request and payment of the necessary fee.

FIG. 1 depicts the empty lentivector into which the CAR construct is cloned. Shown is the 5′ Long Terminal Repeat (5′ LTR) of HIV_{NL4-3}, the Rev-Response Element (RRE), central polypurine tract (cPPT), the CMV enhancer/promoter driving transcription of our CAR and effectors, and the 3′ Self INactivating LTR (3′ SIN LTR), as well as canonical plasmid sequences, including an origin of replication (ori) and ampicillin resistance (AmpR).

FIG. 2 depicts the third generation CAR fused to a proprietary scFv, derived from VAC69, which targets a cancer cell antigen. Other factors are also expressed downstream of an rdm3 Internal Ribosome Entry Site (IRES), but upstream of a Gaussia Dura Luc signal peptide (SP), which mediates secretion. Among these factors are interleukin 7 (IL-7), IL-21, pro-Matrix Metalloproteinase 8 (Pro-MMP8), CXCR3, peptide P60, dominant-negative (DN) Fas-Associated Death Domain (FADD), a Bi-specific T cell Engager (BiTE) based on the VAC69 anticancer scFv and the humanized OKT (hOKT3) scFv, and the puromycin resistance gene (PuroR). Also indicated are transmembrane (TM) domains, glycine-glycine-glycine-serine (GGGS) linkers, and restriction endonuclease sites for removal and/or replacement of individual effectors.

FIG. 3 depicts an alternative embodiment of the third generation CAR fused to an scFv fragment derived from monoclonal antibody VAC 69.

FIG. 4 shows the nucleotide sequence of the alternative embodiment of the third generation CAR fused to an scFv fragment derived from monoclonal antibody VAC 69 depicted in FIG. 3.

FIG. 5 shows the location of the nucleotide sequences and the corresponding amino acid sequences of VAC69 CAR, GSG-P2A Site 1, human serum albumin signal peptide 2, VAC69 BiTE, V5 epitope tag, GSG-P2A Site 2, human serum albumin signal peptide 3, pro-MMP 8, GSG-P2A Site 3, human serum albumin signal peptide 4, IL-7, GSG-P2A Site 4, human serum albumin signal peptide 5, PD-1 Extracellular Domain/4-1BB Transmembrane Domain and Intracellular Domain Fusion, GSG-P2A Site 5, CXCR3, GSG-P2A Site 6, human serum albumin signal peptide 6, and IL 21, of the alternative embodiment of the third generation CAR fused to an scFv fragment derived from monoclonal antibody VAC 69 depicted in FIG. 3.

FIG. 6A-6E is an analysis of expression of factors encoded by the VAC69 CAR vector in HEK293T cells. FIG. 6A shows the results of an immunoblot analysis of expression of VAC69 BiTE. FIG. 6B shows the results of an immunoblot analysis of expression of MMP8. FIG. 6C shows the results of an immunoblot analysis of expression of IL-7. FIG. 6D shows the results of an immunoblot analysis of expression of VAC69 CAR. FIG. 6E shows the results of a flow cytometry analysis of expression of CXCR3 and IL 21.

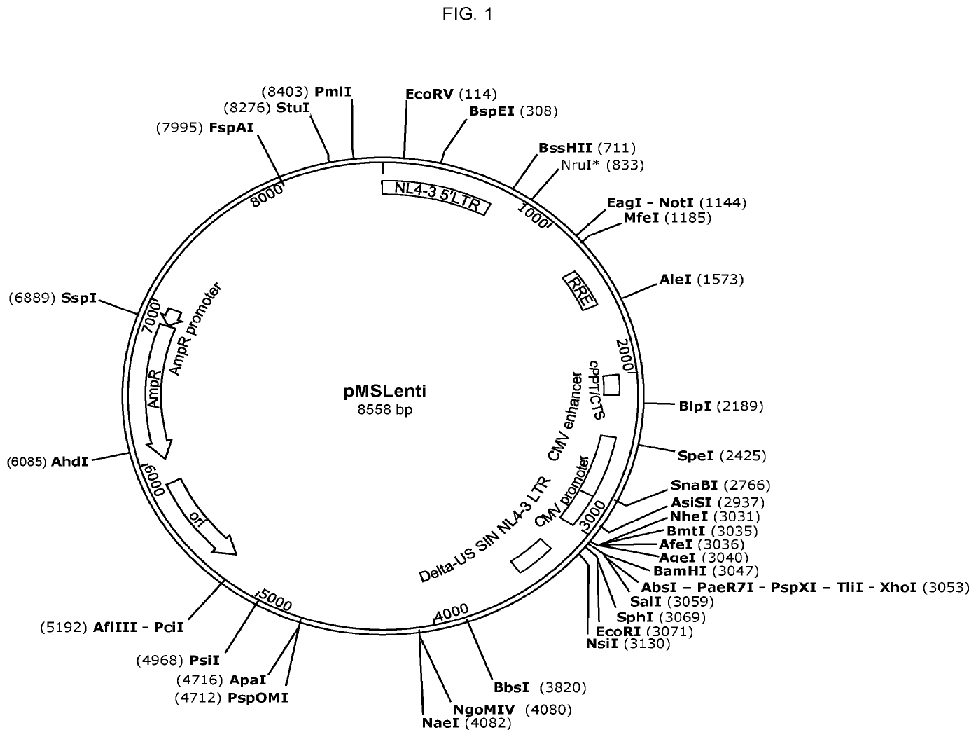

### DETAILED DESCRIPTION

Described herein is a CAR T cell construct encoding multiple factors mediating efficient tumor cytolysis in vivo. These factors include cytokines, chimeric immune receptors, chemokine receptors, and cancer desmoplastic stroma-directed proteases. In some embodiments, this technology refers to the vector design, or to the combination of factors, in part or in whole, mediating efficient tumor targeting and elimination; particularly, in the context of CAR-based therapies.

Described below are the vector and a list of these factors, though not representing every potential instantiation of the technology.

CARs have undergone multiple iterations, representing stepwise improvements is their functionality, and collectively denoted first, second, and third generation CARs. First generation CARs were single-chain Fv (scFv) fusions with either CD3ζ or FcRγ [2, 3]. While promoting cell activation, without co-stimulation, the first generation CARs often resulted in T cell anergy, poor effector functions, and poor persistence [2, 3]. Second generation CARs included CD3ζ along with costimulatory domains from either CD28, OX40, or 4-1BB: these resulted in better effector functions and anti-cancer responses [2, 3]. Third generation CARs include an additional costimulatory domain from either OX40 or 4-1BB in addition to CD28 and the activating CD3ζ intracellular domain (ICD) [2, 3].

CAR T cells have exhibited enormous potential as anti-cancer therapies, particularly among CD19-positive hematological malignancies. In these cases, second and third-generation CAR T cells promote rapid tumor cytolysis in vitro and in patients. However, the effectuality of CAR T cells for solid tumors remains limited [4]. This can be attributed to a number of physical and physiological barriers to immune cell function in the tumor microenvironment [reviewed in 5].

Among the first barriers encountered by tumor-specific T cells is the desmoplastic stroma, a dense mesh of Cancer Associated Fibroblasts (CAFs), along with the Collagen Types I, III, and IV that they secrete, and other proteoglycans and glycosaminoglycans [5, 6]. To facilitate CAR T cell infiltration into solid tumors, the expression of pro-MMP-8 was selected due to its unique properties, which include the following: MMP-8 is specific to collagens Type I, II, and III, but does not display activity to Type IV collagen; while lacking activity to Type IV collagen, MMP-8 does not promote angiogenesis or metastasis, unlike the collagen Type IV-specific MMP-9 [7]; and, lastly, MMP-8 activity inhibits metastasis [8] and promotes the release extracellular matrix (ECM)-derived Proline-Glycine-Proline (PGP), which drives CXCR-dependent neutrophil recruitment [9].

Among the physiological barriers to CAR T cell function in the tumor is the profoundly immunosuppressive environment, which includes complex chemokine gradients that can act fugetactically (driving T cells away), active Tregs, and suppressive cytokines and surface proteins. Among the surface proteins that prevent CAR T cell function is tumor-expressed PD-L1, which binds PD-1 on active T cells, downregulating T cell effector functions. To overcome this physiological hurdle, a PD-1 extracellular domain/4-1BB transmembrane and intracellular domain fusion protein are proposed: the engagement of tumor-expressed PD-L1 by this fusion protein results in the accumulation of the 4-1BB costimulatory intracellular domain, effectively converting an immunosuppressive signal into an immunostimulatory one [10].

Another significant issue for CAR T cell function is cell persistence and homeostatic proliferation. In immunocompetent patients, T cell proliferation is regulated by the endogenous levels of common γ_{c} ligands (IL-2, IL-7, IL-15, and IL-21). IL-7, in particular, is responsible for homeostatic proliferation, but its endogenous levels are limited by competition with host T and B cells [11]. To promote persistence and homeostatic proliferation, the CAR T cell vector will also express IL-7. IL-21 will also be expressed, as in vivo persistence is facilitated by its expression [11].

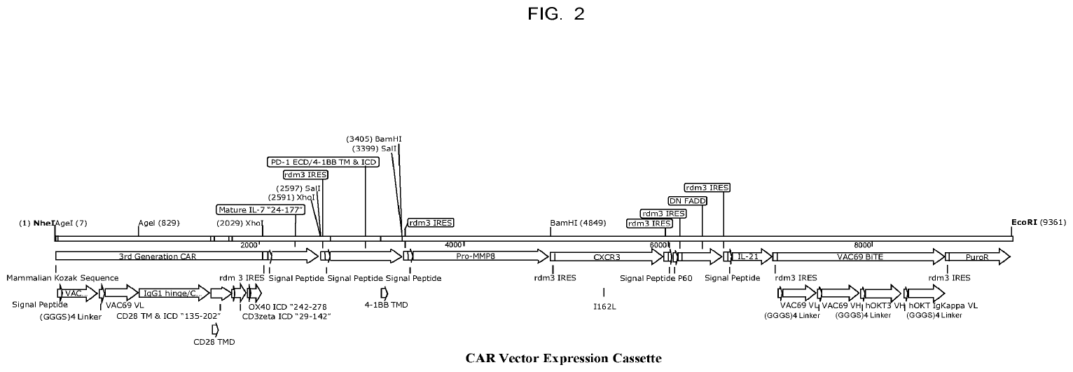

The CAR itself can be a third-generation CD28-CD3ζ-OX40 fusion with the VAC69 scFv region and IgG1 Hinge/CH2/CH3 region (FIG. 2). Also included in FIG. 2, though not addressed in length, are the following: (1) CXCR3 expression and binding to CXCL9 and 10 appears to correlate positively with CD8 T cell tumor infiltration [12]; (2) expression of dominant negative Fas-Associated Death Domain (FADD) may prevent tumor-expressed FasL from inducing cell death in CAR T cells [13]; (3) expression of the FOXP3 inhibitory peptide, P60, which can enter cells, may prevent Treg-mediated immunosuppression of CAR T cells in the tumor microenvironment [14]; and (4) the concomitant expression of a Bi-specific T cell Engager (BiTE) composed of VAC69 scFv, in the order of VL-VH, fused with humanized OKT3 scFV, in the order of VH-VL, which can promote a broader T cell response that includes endogenous host T cells [15].

In one aspect, described herein is a list of factors expressed from a vector, including the following:

a. Interleukin-7 (IL-7);

b. Interleukin-21 (IL-21);

c. Pro-Matrix Metalloproteinase 8 (Pro-MMP-8);

In some embodiments, this includes the following:

a. The expression of other Matrix Metalloproteinases in the context of CAR-based therapies;

b. The expression of any protease in CAR-based therapies.

c. A protein fusion of PD-1 extracellular domain (ECD) and the transmembrane domain (TMD) and intracellular domain (ICD) of 4-1BB;

In some embodiments, this includes the following:

a. The expression of PD-1 fused with any costimulatory protein (e.g. CD28, OX40, or ICOS) among one or more other factors included in this list;

b. The expression of immunosuppressive or immunotoxic surface receptors fused with a costimulatory domain among one or more other factors included in this list;

c. The expression of dominant-negative or nonfunctional immunosuppressive or immunotoxic receptors among one or more other factors included in this list.

d. The expression of chemokine receptor, CXCR3;

e. The expression of a Bi-specific T cell Engager (BiTE) from the same construct as a CAR.

In some embodiments, this includes the following:

a. The expression of any chemokine receptors among one or more other factors included in this list;

b. The expression of other chemotactic receptors (e.g. ROBO1) among one or more other factors included in this list.

c. The expression of dominant-negative Fas-Associate Death Domain (FADD);

d. The expression of FOXP3 inhibitory peptide, P60;

e. The expression of a Bi-specific T cell Engager (BiTE) from the same construct as a CAR.

In some embodiments, this technology constitutes the expression of all components in this list.

In some embodiments, this technology constitutes the expression of a subordinate list of such factors in the context of CAR-based therapies, including:

a. 2 or more of the above-listed factors, excluding IL-7 and IL-21 alone;

b. 3 or move of the above-listed factors;

c. 4 or more of the above-listed factors;

d. 5 or more of the above-listed factors;

e. 6 or more of the above-listed factors;

f. 7 or more of the above-listed factors.

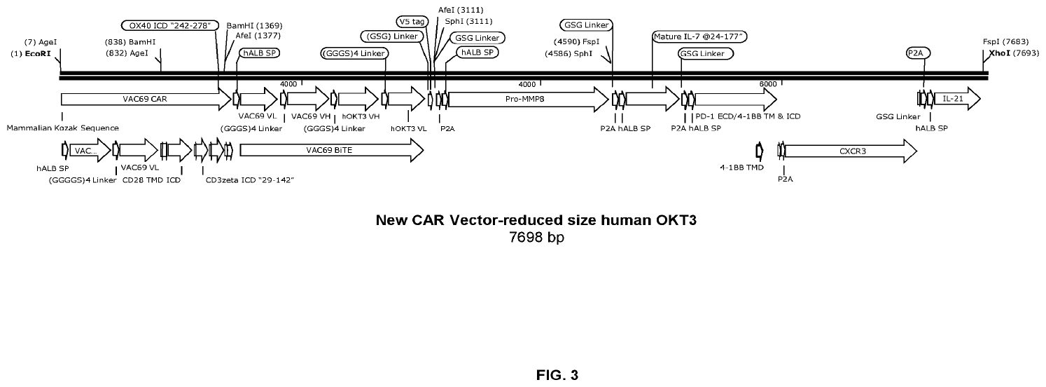

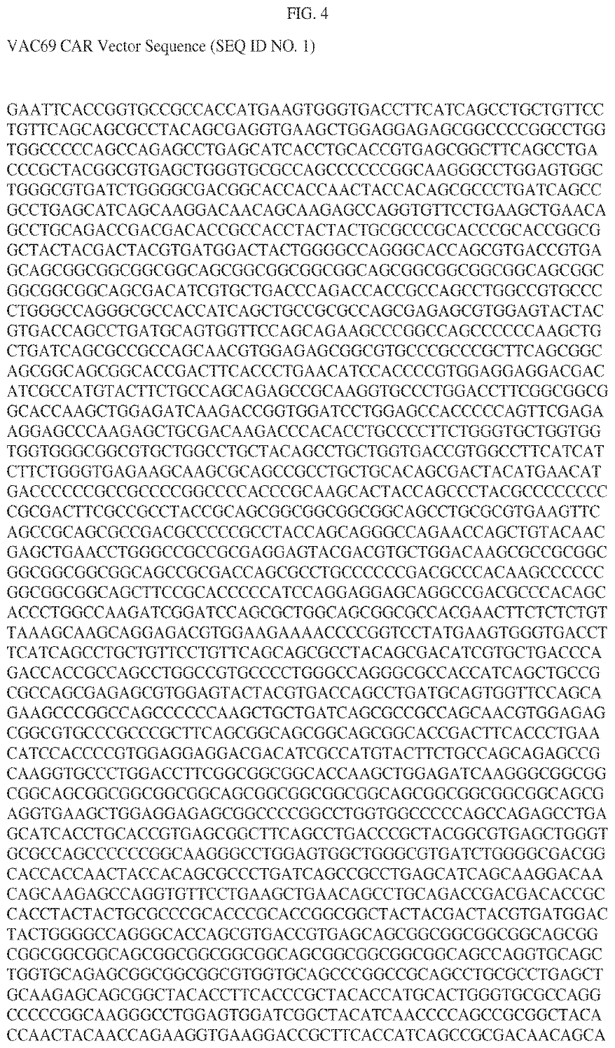

Disclosed herein are CAR constructs that are useful for mediating efficient cytolysis of solid tumors. The constructs include third-generation CARs comprising the VAC69 single-chain Fv (scFv) and the IgG1 heavy chain constant regions CH2 and CH3 fused to the the transmembrane domain (TMD) and intracellular domain (ICD) of CD28, the ICD of CD3ζ, and the ICD of OX40. An exemplary nucleic acid construct is shown in FIG. 2. The construct can include a chimeric antigen receptor (CAR) comprising a single chain variable fragment from monoclonal antibody VAC69; a cytokine; and one or more of the following factors: i) a matrix metaloproteinase; ii) a PD1 fusion protein; iii) a chemokine receptor; iv) a dominant negative or nonfunctional immunosuppressive or toxic receptor; v) a FOXP3 inhibitory peptide P60; and vi) a Bi-specific T Cell Engager (BiTE). An alternative embodiment of an exemplary construct is shown in FIG. 3.

The component factors comprising the constructs can be arranged sequentially as shown in FIG. 2 or FIG. 3. The constructs are not so limited however and the component factors can be arranged in any order provides for expression of the factors.

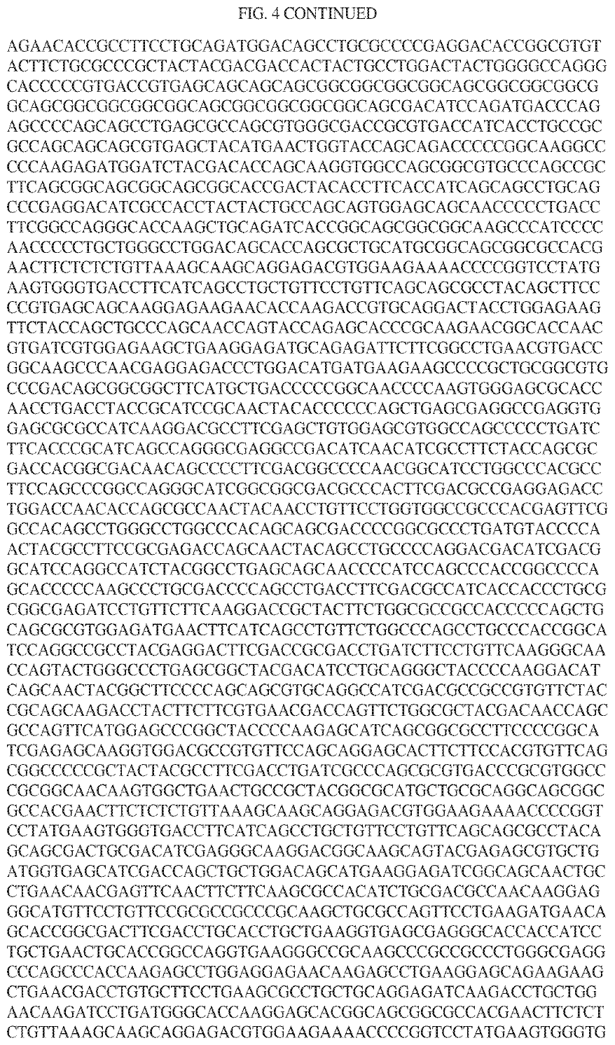

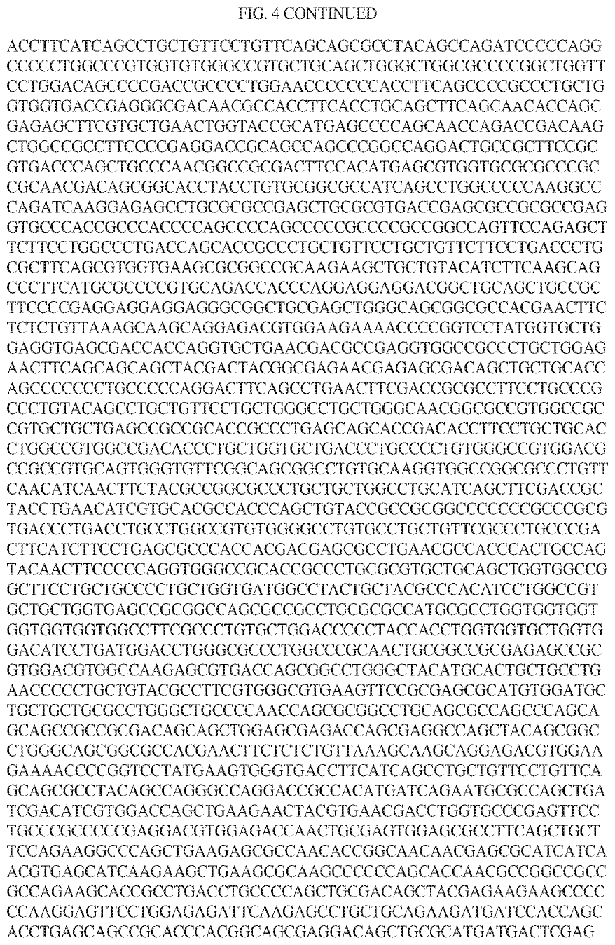

We may use the terms “nucleic acid” and “polynucleotide” interchangeably to refer to both RNA and DNA, including cDNA, genomic DNA, synthetic DNA, and DNA (or RNA) containing nucleic acid analogs, any of which may encode a polypeptide of the invention and all of which are encompassed by the invention. Polynucleotides can have essentially any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode any of the constructs disclosed herein, for example, the constructs shown in FIG. 2, FIG. 3 and FIG. 4, as well as sequences of the individual factors that comprising the constructs.

An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by in several ways. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Two nucleic acids or the polypeptides they encode may be described as having a certain degree of identity to one another. For example, polypeptide and a biologically active variant thereof may be described as exhibiting a certain degree of identity. Alignments may be assembled by locating short sequences in the Protein Information Research (PIR) site (http://pir.georgetown.edu), followed by analysis with the “short nearly identical sequences” Basic Local Alignment Search Tool (BLAST) algorithm on the NCBI website (http://www.ncbi.nlm.nih.gov/blast).

As used herein, the term “percent sequence identity” refers to the degree of identity between any given query sequence and a subject sequence. For example, any of the sequences comprising the constructs disclosed herein disclosed herein can be the query sequence and any of the sequences comprising the constructs disclosed herein disclosed herein can be the subject sequence. Similarly, any of the sequences comprising the constructs disclosed herein disclosed herein can be the query sequence and a biologically active variant thereof can be the subject sequence.

To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment).

ClustalW calculates the best match between a query and one or more subject sequences and aligns them so that identities, similarities and differences can be determined. Gaps of one or more residues can be inserted into a query sequence, a subject sequence, or both, to maximize sequence alignments. For fast pair wise alignment of nucleic acid sequences, the following default parameters are used: word size: 2; window size: 4; scoring method: percentage; number of top diagonals: 4; and gap penalty: 5. For multiple alignments of nucleic acid sequences, the following parameters are used: gap opening penalty: 10.0; gap extension penalty: 5.0; and weight transitions: For fast pair wise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The nucleic acids and polypeptides described herein may be referred to as “exogenous”. The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

Nucleic acids of the invention, that is, nucleic acids having a nucleotide sequence of any of the constructs or component factors disclosed herein, can include nucleic acids sequences that are at least about 70%, at least about 75%, at least about 80%, at least about 85%, at least about 90%, at least about 95%, at least about 96%, at least about 97%, at least about 98%, at least about 99% identical to the sequences disclosed herein.

Thus in some embodiments, the construct can have the nucleic acid sequence of SEQ ID NO. 1 as shown below. In some embodiments the construct can have a nucleic acid sequence that is at least about 70%, at least about 75%, at least about 80%, at least about 85%, at least about 90%, at least about 95%, at least about 96%, at least about 97%, at least about 98%, at least about 99% identical to SEQ ID NO. 1.

VAC69 CAR Vector Sequence
(SEQ ID NO. 1)
GAATTCACCGGTGCCGCCACCATGAAGTGGGTGACCTTCATCAGCCTGC
TGTTCCTGTTCAGCAGCGCCTACAGCGAGGTGAAGCTGGAGGAGAGCGG
CCCCGGCCTGGTGGCCCCCAGCCAGAGCCTGAGCATCACCTGCACCGTG
AGCGGCTTCAGCCTGACCCGCTACGGCGTGAGCTGGGTGCGCCAGCCCC
CCGGCAAGGGCCTGGAGTGGCTGGGCGTGATCTGGGGCGACGGCACCAC
CAACTACCACAGCGCCCTGATCAGCCGCCTGAGCATCAGCAAGGACAAC
AGCAAGAGCCAGGTGTTCCTGAAGCTGAACAGCCTGCAGACCGACGACA
CCGCCACCTACTACTGCGCCCGCACCCGCACCGGCGGCTACTACGACTA
CGTGATGGACTACTGGGGCCAGGGCACCAGCGTGACCGTGAGCAGCGGC
GGCGGCGGCAGCGGCGGCGGCGGCAGCGGCGGCGGCGGCAGCGGCGGCG
GCGGCAGCGACATCGTGCTGACCCAGACCACCGCCAGCCTGGCCGTGCC
CCTGGGCCAGGGCGCCACCATCAGCTGCCGCGCCAGCGAGAGCGTGGAG
TACTACGTGACCAGCCTGATGCAGTGGTTCCAGCAGAAGCCCGGCCAGC
CCCCCAAGCTGCTGATCAGCGCCGCCAGCAACGTGGAGAGCGGCGTGCC
CGCCCGCTTCAGCGGCAGCGGCAGCGGCACCGACTTCACCCTGAACATC
CACCCCGTGGAGGAGGACGACATCGCCATGTACTTCTGCCAGCAGAGCC
GCAAGGTGCCCTGGACCTTCGGCGGCGGCACCAAGCTGGAGATCAAGAC
CGGTGGATCCTGGAGCCACCCCCAGTTCGAGAAGGAGCCCAAGAGCTGC
GACAAGACCCACACCTGCCCCTTCTGGGTGCTGGTGGTGGTGGGCGGCG
TGCTGGCCTGCTACAGCCTGCTGGTGACCGTGGCCTTCATCATCTTCTG
GGTGAGAAGCAAGCGCAGCCGCCTGCTGCACAGCGACTACATGAACATG
ACCCCCCGCCGCCCCGGCCCCACCCGCAAGCACTACCAGCCCTACGCCC
CCCCCCGCGACTTCGCCGCCTACCGCAGCGGCGGCGGCGGCAGCCTGCG
CGTGAAGTTCAGCCGCAGCGCCGACGCCCCCGCCTACCAGCAGGGCCAG
AACCAGCTGTACAACGAGCTGAACCTGGGCCGCCGCGAGGAGTACGACG
TGCTGGACAAGCGCCGCGGCGGCGGCGGCGGCAGCCGCGACCAGCGCCT
GCCCCCCGACGCCCACAAGCCCCCCGGCGGCGGCAGCTTCCGCACCCCC
ATCCAGGAGGAGCAGGCCGACGCCCACAGCACCCTGGCCAAGATCGGAT
CCAGCGCTGGCAGCGGCGCCACGAACTTCTCTCTGTTAAAGCAAGCAGG
AGACGTGGAAGAAAACCCCGGTCCTATGAAGTGGGTGACCTTCATCAGC
CTGCTGTTCCTGTTCAGCAGCGCCTACAGCGACATCGTGCTGACCCAGA
CCACCGCCAGCCTGGCCGTGCCCCTGGGCCAGGGCGCCACCATCAGCTG
CCGCGCCAGCGAGAGCGTGGAGTACTACGTGACCAGCCTGATGCAGTGG
TTCCAGCAGAAGCCCGGCCAGCCCCCCAAGCTGCTGATCAGCGCCGCCA
GCAACGTGGAGAGCGGCGTGCCCGCCCGCTTCAGCGGCAGCGGCAGCGG
CACCGACTTCACCCTGAACATCCACCCCGTGGAGGAGGACGACATCGCC
ATGTACTTCTGCCAGCAGAGCCGCAAGGTGCCCTGGACCTTCGGCGGCG
GCACCAAGCTGGAGATCAAGGGCGGCGGCGGCAGCGGCGGCGGCGGCAG
CGGCGGCGGCGGCAGCGGCGGCGGCGGCAGCGAGGTGAAGCTGGAGGAG
AGCGGCCCCGGCCTGGTGGCCCCCAGCCAGAGCCTGAGCATCACCTGCA
CCGTGAGCGGCTTCAGCCTGACCCGCTACGGCGTGAGCTGGGTGCGCCA
GCCCCCCGGCAAGGGCCTGGAGTGGCTGGGCGTGATCTGGGGCGACGGC
ACCACCAACTACCACAGCGCCCTGATCAGCCGCCTGAGCATCAGCAAGG
ACAACAGCAAGAGCCAGGTGTTCCTGAAGCTGAACAGCCTGCAGACCGA
CGACACCGCCACCTACTACTGCGCCCGCACCCGCACCGGCGGCTACTAC
GACTACGTGATGGACTACTGGGGCCAGGGCACCAGCGTGACCGTGAGCA
GCGGCGGCGGCGGCAGCGGCGGCGGCGGCAGCGGCGGCGGCGGCAGCGG
CGGCGGCGGCAGCCAGGTGCAGCTGGTGCAGAGCGGCGGCGGCGTGGTG
CAGCCCGGCCGCAGCCTGCGCCTGAGCTGCAAGAGCAGCGGCTACACCT
TCACCCGCTACACCATGCACTGGGTGCGCCAGGCCCCCGGCAAGGGCCT
GGAGTGGATCGGCTACATCAACCCCAGCCGCGGCTACACCAACTACAAC
CAGAAGGTGAAGGACCGCTTCACCATCAGCCGCGACAACAGCAAGAACA
CCGCCTTCCTGCAGATGGACAGCCTGCGCCCCGAGGACACCGGCGTGTA
CTTCTGCGCCCGCTACTACGACGACCACTACTGCCTGGACTACTGGGGC
CAGGGCACCCCCGTGACCGTGAGCAGCAGCAGCGGCGGCGGCGGCAGCG
GCGGCGGCGGCAGCGGCGGCGGCGGCAGCGGCGGCGGCGGCAGCGACAT
CCAGATGACCCAGAGCCCCAGCAGCCTGAGCGCCAGCGTGGGCGACCGC
GTGACCATCACCTGCCGCGCCAGCAGCAGCGTGAGCTACATGAACTGGT
ACCAGCAGACCCCCGGCAAGGCCCCCAAGAGATGGATCTACGACACCAG
CAAGGTGGCCAGCGGCGTGCCCAGCCGCTTCAGCGGCAGCGGCAGCGGC
ACCGACTACACCTTCACCATCAGCAGCCTGCAGCCCGAGGACATCGCCA
CCTACTACTGCCAGCAGTGGAGCAGCAACCCCCTGACCTTCGGCCAGGG
CACCAAGCTGCAGATCACCGGCAGCGGCGGCAAGCCCATCCCCAACCCC
CTGCTGGGCCTGGACAGCACCAGCGCTGCATGCGGCAGCGGCGCCACGA
ACTTCTCTCTGTTAAAGCAAGCAGGAGACGTGGAAGAAAACCCCGGTCC
TATGAAGTGGGTGACCTTCATCAGCCTGCTGTTCCTGTTCAGCAGCGCC
TACAGCTTCCCCGTGAGCAGCAAGGAGAAGAACACCAAGACCGTGCAGG
ACTACCTGGAGAAGTTCTACCAGCTGCCCAGCAACCAGTACCAGAGCAC
CCGCAAGAACGGCACCAACGTGATCGTGGAGAAGCTGAAGGAGATGCAG
AGATTCTTCGGCCTGAACGTGACCGGCAAGCCCAACGAGGAGACCCTGG
ACATGATGAAGAAGCCCCGCTGCGGCGTGCCCGACAGCGGCGGCTTCAT
GCTGACCCCCGGCAACCCCAAGTGGGAGCGCACCAACCTGACCTACCGC
ATCCGCAACTACACCCCCCAGCTGAGCGAGGCCGAGGTGGAGCGCGCCA
TCAAGGACGCCTTCGAGCTGTGGAGCGTGGCCAGCCCCCTGATCTTCAC
CCGCATCAGCCAGGGCGAGGCCGACATCAACATCGCCTTCTACCAGCGC
GACCACGGCGACAACAGCCCCTTCGACGGCCCCAACGGCATCCTGGCCC
ACGCCTTCCAGCCCGGCCAGGGCATCGGCGGCGACGCCCACTTCGACGC
CGAGGAGACCTGGACCAACACCAGCGCCAACTACAACCTGTTCCTGGTG
GCCGCCCACGAGTTCGGCCACAGCCTGGGCCTGGCCCACAGCAGCGACC
CCGGCGCCCTGATGTACCCCAACTACGCCTTCCGCGAGACCAGCAACTA
CAGCCTGCCCCAGGACGACATCGACGGCATCCAGGCCATCTACGGCCTG
AGCAGCAACCCCATCCAGCCCACCGGCCCCAGCACCCCCAAGCCCTGCG
ACCCCAGCCTGACCTTCGACGCCATCACCACCCTGCGCGGCGAGATCCT
GTTCTTCAAGGACCGCTACTTCTGGCGCCGCCACCCCCAGCTGCAGCGC
GTGGAGATGAACTTCATCAGCCTGTTCTGGCCCAGCCTGCCCACCGGCA
TCCAGGCCGCCTACGAGGACTTCGACCGCGACCTGATCTTCCTGTTCAA
GGGCAACCAGTACTGGGCCCTGAGCGGCTACGACATCCTGCAGGGCTAC
CCCAAGGACATAGCAACTACGGCTTCCCCAGCAGCGTGCAGGCCATCGA
CGCCGCCGTGTTCTACCGCAGCAAGACCTACTTCTTCGTGAACGACCAG
TTCTGGCGCTACGACAACCAGCGCCAGTTCATGGAGCCCGGCTACCCCA
AGAGCATCAGCGGCGCCTTCCCCGGCATCGAGAGCAAGGTGGACGCCGT
GTTCCAGCAGGAGCACTTCTTCCACGTGTTCAGCGGCCCCCGCTACTAC
GCCTTCGACCTGATCGCCCAGCGCGTGACCCGCGTGGCCCGCGGCAACA
AGTGGCTGAACTGCCGCTACGGCGCATGCTGCGCAGGCAGCGGCGCCAC
GAACTTCTCTCTGTTAAAGCAAGCAGGAGACGTGGAAGAAAACCCCGGT
CCTATGAAGTGGGTGACCTTCATCAGCCTGCTGTTCCTGTTCAGCAGCG
CCTACAGCAGCGACTGCGACATCGAGGGCAAGGACGGCAAGCAGTACGA
GAGCGTGCTGATGGTGAGCATCGACCAGCTGCTGGACAGCATGAAGGAG
ATCGGCAGCAACTGCCTGAACAACGAGTTCAACTTCTTCAAGCGCCACA
TCTGCGACGCCAACAAGGAGGGCATGTTCCTGTTCCGCGCCGCCCGCAA
GCTGCGCCAGTTCCTGAAGATGAACAGCACCGGCGACTTCGACCTGCAC
CTGCTGAAGGTGAGCGAGGGCACCACCATCCTGCTGAACTGCACCGGCC
AGGTGAAGGGCCGCAAGCCCGCCGCCCTGGGCGAGGCCCAGCCCACCAA
GAGCCTGGAGGAGAACAAGAGCCTGAAGGAGCAGAAGAAGCTGAACGAC
CTGTGCTTCCTGAAGCGCCTGCTGCAGGAGATCAAGACCTGCTGGAACA
AGATCCTGATGGGCACCAAGGAGCACGGCAGCGGCGCCACGAACTTCTC
TCTGTTAAAGCAAGCAGGAGACGTGGAAGAAAACCCCGGTCCTATGAAG
TGGGTGACCTTCATCAGCCTGCTGTTCCTGTTCAGCAGCGCCTACAGCC
AGATCCCCCAGGCCCCCTGGCCCGTGGTGTGGGCCGTGCTGCAGCTGGG
CTGGCGCCCCGGCTGGTTCCTGGACAGCCCCGACCGCCCCTGGAACCCC
CCCACCTTCAGCCCCGCCCTGCTGGTGGTGACCGAGGGCGACAACGCCA
CCTTCACCTGCAGCTTCAGCAACACCAGCGAGAGCTTCGTGCTGAACTG
GTACCGCATGAGCCCCAGCAACCAGACCGACAAGCTGGCCGCCTTCCCC
GAGGACCGCAGCCAGCCCGGCCAGGACTGCCGCTTCCGCGTGACCCAGC
TGCCCAACGGCCGCGACTTCCACATGAGCGTGGTGCGCGCCCGCCGCAA
CGACAGCGGCACCTACCTGTGCGGCGCCATCAGCCTGGCCCCCAAGGCC
CAGATCAAGGAGAGCCTGCGCGCCGAGCTGCGCGTGACCGAGCGCCGCG
CCGAGGTGCCCACCGCCCACCCCAGCCCCAGCCCCCGCCCCGCCGGCCA
GTTCCAGAGCTTCTTCCTGGCCCTGACCAGCACCGCCCTGCTGTTCCTG
CTGTTCTTCCTGACCCTGCGCTTCAGCGTGGTGAAGCGCGGCCGCAAGA
AGCTGCTGTACATCTTCAAGCAGCCCTTCATGCGCCCCGTGCAGACCAC
CCAGGAGGAGGACGGCTGCAGCTGCCGCTTCCCCGAGGAGGAGGAGGGC
GGCTGCGAGCTGGGCAGCGGCGCCACGAACTTCTCTCTGTTAAAGCAAG
CAGGAGACGTGGAAGAAAACCCCGGTCCTATGGTGCTGGAGGTGAGCGA
CCACCAGGTGCTGAACGACGCCGAGGTGGCCGCCCTGCTGGAGAACTTC
AGCAGCAGCTACGACTACGGCGAGAACGAGAGCGACAGCTGCTGCACCA
GCCCCCCCTGCCCCCAGGACTTCAGCCTGAACTTCGACCGCGCCTTCCT
GCCCGCCCTGTACAGCCTGCTGTTCCTGCTGGGCCTGCTGGGCAACGGC
GCCGTGGCCGCCGTGCTGCTGAGCCGCCGCACCGCCCTGAGCAGCACCG
ACACCTTCCTGCTGCACCTGGCCGTGGCCGACACCCTGCTGGTGCTGAC
CCTGCCCCTGTGGGCCGTGGACGCCGCCGTGCAGTGGGTGTTCGGCAGC
GGCCTGTGCAAGGTGGCCGGCGCCCTGTTCAACATCAACTTCTACGCCG
GCGCCCTGCTGCTGGCCTGCATCAGCTTCGACCGCTACCTGAACATCGT
GCACGCCACCCAGCTGTACCGCCGCGGCCCCCCCGCCCGCGTGACCCTG
ACCTGCCTGGCCGTGTGGGGCCTGTGCCTGCTGTTCGCCCTGCCCGACT
TCATCTTCCTGAGCGCCCACCACGACGAGCGCCTGAACGCCACCCACTG
CCAGTACAACTTCCCCCAGGTGGGCCGCACCGCCCTGCGCGTGCTGCAG
CTGGTGGCCGGCTTCCTGCTGCCCCTGCTGGTGATGGCCTACTGCTACG
CCCACATCCTGGCCGTGCTGCTGGTGAGCCGCGGCCAGCGCCGCCTGCG
CGCCATGCGCCTGGTGGTGGTGGTGGTGGTGGCCTTCGCCCTGTGCTGG
ACCCCCTACCACCTGGTGGTGCTGGTGGACATCCTGATGGACCTGGGCG
CCCTGGCCCGCAACTGCGGCCGCGAGAGCCGCGTGGACGTGGCCAAGAG
CGTGACCAGCGGCCTGGGCTACATGCACTGCTGCCTGAACCCCCTGCTG
TACGCCTTCGTGGGCGTGAAGTTCCGCGAGCGCATGTGGATGCTGCTGC
TGCGCCTGGGCTGCCCCAACCAGCGCGGCCTGCAGCGCCAGCCCAGCAG
CAGCCGCCGCGACAGCAGCTGGAGCGAGACCAGCGAGGCCAGCTACAGC
GGCCTGGGCAGCGGCGCCACGAACTTCTCTCTGTTAAAGCAAGCAGGAG
ACGTGGAAGAAAACCCCGGTCCTATGAAGTGGGTGACCTTCATCAGCCT
GCTGTTCCTGTTCAGCAGCGCCTACAGCCAGGGCCAGGACCGCCACATG
ATCAGAATGCGCCAGCTGATCGACATCGTGGACCAGCTGAAGAACTACG
TGAACGACCTGGTGCCCGAGTTCCTGCCCGCCCCCGAGGACGTGGAGAC
CAACTGCGAGTGGAGCGCCTTCAGCTGCTTCCAGAAGGCCCAGCTGAAG
AGCGCCAACACCGGCAACAACGAGCGCATCATCAACGTGAGCATCAAGA
AGCTGAAGCGCAAGCCCCCCAGCACCAACGCCGGCCGCCGCCAGAAGCA
CCGCCTGACCTGCCCCAGCTGCGACAGCTACGAGAAGAAGCCCCCCAAG
GAGTTCCTGGAGAGATTCAAGAGCCTGCTGCAGAAGATGATCCACCAGC
ACCTGAGCAGCCGCACCCACGGCAGCGAGGACAGCTGCGCATGATGACT
CGAG

The component factors comprising the constructs disclosed here in can have the nucleotide sequence of any of the sequences shown below. Thus the component factors can have the nucleic acid sequence of: SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, or SEQ ID NO. 40. In some embodiments, the component factor can have a nucleic acid sequence that is at least about 70%, at least about 75%, at least about 80%, at least about 85%, at least about 90%, at least about 95%, at least about 96%, at least about 97%, at least about 98%, at least about 99% identical to the nucleic acid sequence of: SEQ ID NO. 2, SEQ ID NO. 4, SEQ ID NO. 6, SEQ ID NO. 8, SEQ ID NO. 10, SEQ ID NO. 12, SEQ ID NO. 14, SEQ ID NO. 16, SEQ ID NO. 16, SEQ ID NO. 18, SEQ ID NO. 20, SEQ ID NO. 22, SEQ ID NO. 24, SEQ ID NO. 26, SEQ ID NO. 28, SEQ ID NO. 30, SEQ ID NO. 32, SEQ ID NO. 34, SEQ ID NO. 36, SEQ ID NO. 38, or SEQ ID NO. 40.

Human Serum Albumin Signal Peptide 1
Nucleotide Location: 22-75
Nucleotide Sequence:
ATGAAGTGGGTGACCTTCATCAGCCTGCTGTTCCTGTTCAGCAGCGCCTACAGC
(SEQ ID NO. 2)
Amino Acid Sequence: MKWVTFISLLFLFSSAYS (SEQ ID NO. 3)
VAC69 CAR
Nucleotide Location: 76-1368
Nucleotide Sequence:
GAGGTGAAGCTGGAGGAGAGCGGCCCCGGCCTGGTGGCCCCCAGCCAGAGCCTG
AGCATCACCTGCACCGTGAGCGGCTTCAGCCTGACCCGCTACGGCGTGAGCTGG
GTGCGCCAGCCCCCCGGCAAGGGCCTGGAGTGGCTGGGCGTGATCTGGGGCGAC
GGCACCACCAACTACCACAGCGCCCTGATCAGCCGCCTGAGCATCAGCAAGGAC
AACAGCAAGAGCCAGGTGTTCCTGAAGCTGAACAGCCTGCAGACCGACGACACC
GCCACCTACTACTGCGCCCGCACCCGCACCGGCGGCTACTACGACTACGTGATGG
ACTACTGGGGCCAGGGCACCAGCGTGACCGTGAGCAGCGGCGGCGGCGGCAGC
GGCGGCGGCGGCAGCGGCGGCGGCGGCAGCGGCGGCGGCGGCAGCGACATCGT
GCTGACCCAGACCACCGCCAGCCTGGCCGTGCCCCTGGGCCAGGGCGCCACCAT
CAGCTGCCGCGCCAGCGAGAGCGTGGAGTACTACGTGACCAGCCTGATGCAGTG
GTTCCAGCAGAAGCCCGGCCAGCCCCCCAAGCTGCTGATCAGCGCCGCCAGCAA
CGTGGAGAGCGGCGTGCCCGCCCGCTTCAGCGGCAGCGGCAGCGGCACCGACTT
CACCCTGAACATCCACCCCGTGGAGGAGGACGACATCGCCATGTACTTCTGCCA
GCAGAGCCGCAAGGTGCCCTGGACCTTCGGCGGCGGCACCAAGCTGGAGATCAA
GACCGGTGGATCCTGGAGCCACCCCCAGTTCGAGAAGGAGCCCAAGAGCTGCGA
CAAGACCCACACCTGCCCCTTCTGGGTGCTGGTGGTGGTGGGCGGCGTGCTGGCC
TGCTACAGCCTGCTGGTGACCGTGGCCTTCATCATCTTCTGGGTGAGAAGCAAGC
GCAGCCGCCTGCTGCACAGCGACTACATGAACATGACCCCCCGCCGCCCCGGCC
CCACCCGCAAGCACTACCAGCCCTACGCCCCCCCCCGCGACTTCGCCGCCTACCG
CAGCGGCGGCGGCGGCAGCCTGCGCGTGAAGTTCAGCCGCAGCGCCGACGCCCC
CGCCTACCAGCAGGGCCAGAACCAGCTGTACAACGAGCTGAACCTGGGCCGCCG
CGAGGAGTACGACGTGCTGGACAAGCGCCGCGGCGGCGGCGGCGGCAGCCGCG
ACCAGCGCCTGCCCCCCGACGCCCACAAGCCCCCCGGCGGCGGCAGCTTCCGCA
CCCCCATCCAGGAGGAGCAGGCCGACGCCCACAGCACCCTGGCCAAGATCGGAT
CCAGCGCT (SEQ ID NO. 4)
Amino Acid Sequence:
EVKLEESGPGLVAPSQSLSITCTVSGFSLTRYGVSWVRQPPGKGLEWLGVIWGDGTT
NYHSALISRLSISKDNSKSQVFLKLNSLQTDDTATYYCARTRTGGYYDYVMDYWGQ
GTSVTVSSGGGGSGGGGSGGGGSGGGGSDIVLTQTTASLAVPLGQGATISCRASESV
EYYVTSLMQWFQQKPGQPPKLLISAASNVESGVPARFSGSGSGTDFTLNIHPVEEDDI
AMYFCQQSRKVPWTFGGGTKLEIKTGGSWSHPQFEKEPKSCDKTHTCPFWVLVVVG
GVLACYSLLVTVAFIIFWVRSKRSRLLHSDYMNMTPRRPGPTRKHYQPYAPPRDFAA
YRSGGGGSLRVKFSRSADAPAYQQGQNQLYNELNLGRREEYDVLDKRRGGGGGSR
DQRLPPDAHKPPGGGSFRTPIQEEQADAHSTLAKIGSSA (SEQ ID NO. 5)
GSG-P2A Site 1
Nucleotide Location: 1381-1446
Nucleotide Sequence:
GGCAGCGGCGCCACGAACTTCTCTCTGTTAAAGCAAGCAGGAGACGTGGAAGAA
AACCCCGGTCCT (SEQ ID NO. 6)
Amino Acid Sequence: GSGATNFSLLKQAGDVEENPGP (SEQ ID NO. 7)
Human Serum Albumin Signal Peptide 2
Nucleotide Location: 1447-1500
Nucleotide Sequence:
ATGAAGTGGGTGACCTTCATCAGCCTGCTGTTCCTGTTCAGCAGCGCCTACAGC
(SEQ ID NO. 8)
Amino Acid Sequence: MKWVTFISLLFLFSSAYS (SEQ ID NO. 9)
VAC69 BiTE
Nucleotide Location: 1501-3057
Nucleotide Sequence:
GACATCGTGCTGACCCAGACCACCGCCAGCCTGGCCGTGCCCCTGGGCCAGGGC
GCCACCATCAGCTGCCGCGCCAGCGAGAGCGTGGAGTACTACGTGACCAGCCTG
ATGCAGTGGTTCCAGCAGAAGCCCGGCCAGCCCCCCAAGCTGCTGATCAGCGCC
GCCAGCAACGTGGAGAGCGGCGTGCCCGCCCGCTTCAGCGGCAGCGGCAGCGGC
ACCGACTTCACCCTGAACATCCACCCCGTGGAGGAGGACGACATCGCCATGTAC
TTCTGCCAGCAGAGCCGCAAGGTGCCCTGGACCTTCGGCGGCGGCACCAAGCTG
GAGATCAAGGGCGGCGGCGGCAGCGGCGGCGGCGGCAGCGGCGGCGGCGGCAG
CGGCGGCGGCGGCAGCGAGGTGAAGCTGGAGGAGAGCGGCCCCGGCCTGGTGG
CCCCCAGCCAGAGCCTGAGCATCACCTGCACCGTGAGCGGCTTCAGCCTGACCC
GCTACGGCGTGAGCTGGGTGCGCCAGCCCCCCGGCAAGGGCCTGGAGTGGCTGG
GCGTGATCTGGGGCGACGGCACCACCAACTACCACAGCGCCCTGATCAGCCGCC
TGAGCATCAGCAAGGACAACAGCAAGAGCCAGGTGTTCCTGAAGCTGAACAGCC
TGCAGACCGACGACACCGCCACCTACTACTGCGCCCGCACCCGCACCGGCGGCT
ACTACGACTACGTGATGGACTACTGGGGCCAGGGCACCAGCGTGACCGTGAGCA
GCGGCGGCGGCGGCAGCGGCGGCGGCGGCAGCGGCGGCGGCGGCAGCGGCGGC
GGCGGCAGCCAGGTGCAGCTGGTGCAGAGCGGCGGCGGCGTGGTGCAGCCCGGC
CGCAGCCTGCGCCTGAGCTGCAAGAGCAGCGGCTACACCTTCACCCGCTACACC
ATGCACTGGGTGCGCCAGGCCCCCGGCAAGGGCCTGGAGTGGATCGGCTACATC
AACCCCAGCCGCGGCTACACCAACTACAACCAGAAGGTGAAGGACCGCTTCACC
ATCAGCCGCGACAACAGCAAGAACACCGCCTTCCTGCAGATGGACAGCCTGCGC
CCCGAGGACACCGGCGTGTACTTCTGCGCCCGCTACTACGACGACCACTACTGCC
TGGACTACTGGGGCCAGGGCACCCCCGTGACCGTGAGCAGCAGCAGCGGCGGCG
GCGGCAGCGGCGGCGGCGGCAGCGGCGGCGGCGGCAGCGGCGGCGGCGGCAGC
GACATCCAGATGACCCAGAGCCCCAGCAGCCTGAGCGCCAGCGTGGGCGACCGC
GTGACCATCACCTGCCGCGCCAGCAGCAGCGTGAGCTACATGAACTGGTACCAG
CAGACCCCCGGCAAGGCCCCCAAGAGATGGATCTACGACACCAGCAAGGTGGCC
AGCGGCGTGCCCAGCCGCTTCAGCGGCAGCGGCAGCGGCACCGACTACACCTTC
ACCATCAGCAGCCTGCAGCCCGAGGACATCGCCACCTACTACTGCCAGCAGTGG
AGCAGCAACCCCCTGACCTTCGGCCAGGGCACCAAGCTGCAGATCACC (SEQ ID
NO. 10)
Amino Acid Sequence:
DIVLTQTTASLAVPLGQGATISCRASESVEYYVTSLMQWFQQKPGQPPKLLISAASNV
ESGVPARFSGSGSGTDFTLNIHPVEEDDIAMYFCQQSRKVPWTFGGGTKLEIKGGGG
SGGGGSGGGGSGGGGSEVKLEESGPGLVAPSQSLSITCTVSGFSLTRYGVSWVRQPP
GKGLEWLGVIWGDGTTNYHSALISRLSISKDNSKSQVFLKLNSLQTDDTATYYCART
RTGGYYDYVMDYWGQGTSVTVSSGGGGSGGGGSGGGGSGGGGSQVQLVQSGGGV
VQPGRSLRLSCKSSGYTFTRYTMHWVRQAPGKGLEWIGYINPSRGYTNYNQKVKDR
FTISRDNSKNTAFLQMDSLRPEDTGVYFCARYYDDHYCLDYWGQGTPVTVSSSSGG
GGSGGGGSGGGGSGGGGSDIQMTQSPSSLSASVGDRVTITCRASSSVSYMNWYQQT
PGKAPKRWIYDTSKVASGVPSRFSGSGSGTDYTFTISSLQPEDIATYYCQQWSSNPLT
FGQGTKLQIT (SEQ ID NO. 11)
V5 Epitope Tag
Nucleotide Location: 3067-3108
Nucleotide Sequence:
GGCAAGCCCATCCCCAACCCCCTGCTGGGCCTGGACAGCACC (SEQ ID NO. 12)
Amino Acid Sequence: GKPIPNPLLGLDST (SEQ ID NO. 13)
GSG-P2A Site 2
Nucleotide Location: 3121-3186
Nucleotide Sequence:
GGCAGCGGCGCCACGAACTTCTCTCTGTTAAAGCAAGCAGGAGACGTGGAAGAA
AACCCCGGTCCT (SEQ ID NO. 14)
Amino Acid Sequence: GSGATNFSLLKQAGDVEENPGP (SEQ ID NO. 15)
Human Serum Albumin Signal Peptide 3
Nucleotide Location: 3187-3240
Nucleotide Sequence:
ATGAAGTGGGTGACCTTCATCAGCCTGCTGTTCCTGTTCAGCAGCGCCTACAGC
(SEQ ID NO. 16)
Amino Acid Sequence: MKWVTFISLLFLFSSAYS (SEQ ID NO. 17)
Pro-MMP8
Nucleotide Location: 3241-4581
Nucleotide Sequence:
TTCCCCGTGAGCAGCAAGGAGAAGAACACCAAGACCGTGCAGGACTACCTGGAG
AAGTTCTACCAGCTGCCCAGCAACCAGTACCAGAGCACCCGCAAGAACGGCACC
AACGTGATCGTGGAGAAGCTGAAGGAGATGCAGAGATTCTTCGGCCTGAACGTG
ACCGGCAAGCCCAACGAGGAGACCCTGGACATGATGAAGAAGCCCCGCTGCGGC
GTGCCCGACAGCGGCGGCTTCATGCTGACCCCCGGCAACCCCAAGTGGGAGCGC
ACCAACCTGACCTACCGCATCCGCAACTACACCCCCCAGCTGAGCGAGGCCGAG
GTGGAGCGCGCCATCAAGGACGCCTTCGAGCTGTGGAGCGTGGCCAGCCCCCTG
ATCTTCACCCGCATCAGCCAGGGCGAGGCCGACATCAACATCGCCTTCTACCAGC
GCGACCACGGCGACAACAGCCCCTTCGACGGCCCCAACGGCATCCTGGCCCACG
CCTTCCAGCCCGGCCAGGGCATCGGCGGCGACGCCCACTTCGACGCCGAGGAGA
CCTGGACCAACACCAGCGCCAACTACAACCTGTTCCTGGTGGCCGCCCACGAGTT
CGGCCACAGCCTGGGCCTGGCCCACAGCAGCGACCCCGGCGCCCTGATGTACCC
CAACTACGCCTTCCGCGAGACCAGCAACTACAGCCTGCCCCAGGACGACATCGA
CGGCATCCAGGCCATCTACGGCCTGAGCAGCAACCCCATCCAGCCCACCGGCCC
CAGCACCCCCAAGCCCTGCGACCCCAGCCTGACCTTCGACGCCATCACCACCCTG
CGCGGCGAGATCCTGTTCTTCAAGGACCGCTACTTCTGGCGCCGCCACCCCCAGC
TGCAGCGCGTGGAGATGAACTTCATCAGCCTGTTCTGGCCCAGCCTGCCCACCGG
CATCCAGGCCGCCTACGAGGACTTCGACCGCGACCTGATCTTCCTGTTCAAGGGC
AACCAGTACTGGGCCCTGAGCGGCTACGACATCCTGCAGGGCTACCCCAAGGAC
ATCAGCAACTACGGCTTCCCCAGCAGCGTGCAGGCCATCGACGCCGCCGTGTTCT
ACCGCAGCAAGACCTACTTCTTCGTGAACGACCAGTTCTGGCGCTACGACAACCA
GCGCCAGTTCATGGAGCCCGGCTACCCCAAGAGCATCAGCGGCGCCTTCCCCGG
CATCGAGAGCAAGGTGGACGCCGTGTTCCAGCAGGAGCACTTCTTCCACGTGTTC
AGCGGCCCCCGCTACTACGCCTTCGACCTGATCGCCCAGCGCGTGACCCGCGTGG
CCCGCGGCAACAAGTGGCTGAACTGCCGCTACGGC (SEQ ID NO. 18)
Amino Acid Sequence:
FPVSSKEKNTKTVQDYLEKFYQLPSNQYQSTRKNGTNVIVEKLKEMQRFFGLNVTG
KPNEETLDMMKKPRCGVPDSGGFMLTPGNPKWERTNLTYRIRNYTPQLSEAEVERA
IKDAFELWSVASPLIFTRISQGEADINIAFYQRDHGDNSPFDGPNGILAHAFQPGQGIG
GDAHFDAEETWTNTSANYNLFLVAAHEFGHSLGLAHSSDPGALMYPNYAFRETSNY
SLPQDDIDGIQAIYGLSSNPIQPTGPSTPKPCDPSLTFDAITTLRGEILFFKDRYFWRRH
PQLQRVEMNFISLFWPSLPTGIQAAYEDFDRDLIFLFKGNQYWALSGYDILQGYPKDI
SNYGFPSSVQAIDAAVFYRSKTYFFVNDQFWRYDNQRQFMEPGYPKSISGAFPGIES
KVDAVFQQEHFFHVFSGPRYYAFDLIAQRVTRVARGNKWLNCRYG (SEQ ID NO.
19)
GSG-P2A Site 3
Nucleotide Location: 4594-4659
Nucleotide Sequence:
GGCAGCGGCGCCACGAACTTCTCTCTGTTAAAGCAAGCAGGAGACGTGGAAGAA
AACCCCGGTCCT (SEQ ID NO. 20)
Amino Acid Sequence: GSGATNFSLLKQAGDVEENPGP (SEQ ID NO. 21)
Human Serum Albumin Signal Peptide 4
Nucleotide Location: 4660-4713
Nucleotide Sequence:
ATGAAGTGGGTGACCTTCATCAGCCTGCTGTTCCTGTTCAGCAGCGCCTACAGC
(SEQ ID NO. 22)
Amino Acid Sequence: MKWVTFISLLFLFSSAYS (SEQ ID NO. 23)
IL-7
Nucleotide Location: 4714-5172
Nucleotide Sequence:
AGCGACTGCGACATCGAGGGCAAGGACGGCAAGCAGTACGAGAGCGTGCTGAT
GGTGAGCATCGACCAGCTGCTGGACAGCATGAAGGAGATCGGCAGCAACTGCCT
GAACAACGAGTTCAACTTCTTCAAGCGCCACATCTGCGACGCCAACAAGGAGGG
CATGTTCCTGTTCCGCGCCGCCCGCAAGCTGCGCCAGTTCCTGAAGATGAACAGC
ACCGGCGACTTCGACCTGCACCTGCTGAAGGTGAGCGAGGGCACCACCATCCTG
CTGAACTGCACCGGCCAGGTGAAGGGCCGCAAGCCCGCCGCCCTGGGCGAGGCC
CAGCCCACCAAGAGCCTGGAGGAGAACAAGAGCCTGAAGGAGCAGAAGAAGCT
GAACGACCTGTGCTTCCTGAAGCGCCTGCTGCAGGAGATCAAGACCTGCTGGAA
CAAGATCCTGATGGGCACCAAGGAGCA (SEQ ID NO. 24)
Amino Acid Sequence:
SDCDIEGKDGKQYESVLMVSIDQLLDSMKEIGSNCLNNEFNFFKRHICDANKEGMFL
FRAARKLRQFLKMNSTGDFDLHLLKVSEGTTILLNCTGQVKGRKPAALGEAQPTKSL
EENKSLKEQKKLNDLCFLKRLLQEIKTCWNKILMGTKEH (SEQ ID NO. 25)
GSG-P2A Site 4
Nucleotide Location: 5173-5238
Nucleotide Sequence:
GGCAGCGGCGCCACGAACTTCTCTCTGTTAAAGCAAGCAGGAGACGTGGAAGAA
AACCCCGGTCCT (SEQ ID NO. 26)
Amino Acid Sequence: GSGATNFSLLKQAGDVEENPGP (SEQ ID NO. 27)
Human Serum Albumin Signal Peptide 5
Nucleotide Location: 5239-5292
Nucleotide Sequence:
ATGAAGTGGGTGACCTTCATCAGCCTGCTGTTCCTGTTCAGCAGCGCCTACAGC
(SEQ ID NO. 28)
Amino Acid Sequence: MKWVTFISLLFLFSSAYS (SEQ ID NO. 29)
PD-1 Extracellular Domain/4-1BB Transmembrane Domain and Intracellular Domain Fusion
Nucleotide Location: 5293-5991, 4-1BB start 5797
Nucleotide Sequence:
CAGATCCCCCAGGCCCCCTGGCCCGTGGTGTGGGCCGTGCTGCAGCTGGGCTGGC
GCCCCGGCTGGTTCCTGGACAGCCCCGACCGCCCCTGGAACCCCCCCACCTTCAG
CCCCGCCCTGCTGGTGGTGACCGAGGGCGACAACGCCACCTTCACCTGCAGCTTC
AGCAACACCAGCGAGAGCTTCGTGCTGAACTGGTACCGCATGAGCCCCAGCAAC
CAGACCGACAAGCTGGCCGCCTTCCCCGAGGACCGCAGCCAGCCCGGCCAGGAC
TGCCGCTTCCGCGTGACCCAGCTGCCCAACGGCCGCGACTTCCACATGAGCGTGG
TGCGCGCCCGCCGCAACGACAGCGGCACCTACCTGTGCGGCGCCATCAGCCTGG
CCCCCAAGGCCCAGATCAAGGAGAGCCTGCGCGCCGAGCTGCGCGTGACCGAGC
GCCGCGCCGAGGTGCCCACCGCCCACCCCAGCCCCAGCCCCCGCCCCGCCGGCC
AGTTCCAGAGCTTCTTCCTGGCCCTGACCAGCACCGCCCTGCTGTTCCTGCTGTTC
TTCCTGACCCTGCGCTTCAGCGTGGTGAAGCGCGGCCGCAAGAAGCTGCTGTACA
TCTTCAAGCAGCCCTTCATGCGCCCCGTGCAGACCACCCAGGAGGAGGACGGCT
GCAGCTGCCGCTTCCCCGAGGAGGAGGAGGGCGGCTGCGAGCTG (SEQ ID NO.
30)
Amino Acid Sequence:
QIPQAPWPVVWAVLQLGWRPGWFLDSPDRPWNPPTFSPALLVVTEGDNATFTCSFS
NTSESFVLNWYRMSPSNQTDKLAAFPEDRSQPGQDCRFRVTQLPNGRDFHMSVVRA
RRNDSGTYLCGAISLAPKAQIKESLRAELRVTERRAEVPTAHPSPSPRPAGQFQSFFLA
LTSTALLFLLFFLTLRFSVVKRGRKKLLYIFKQPFMRPVQTTQEEDGCSCRFPEEEEG
GCEL (SEQ ID NO. 31)
GSG-P2A Site 5
Nucleotide Location: 5992-6057
Nucleotide Sequence:
GGCAGCGGCGCCACGAACTTCTCTCTGTTAAAGCAAGCAGGAGACGTGGAAGAA
AACCCCGGTCCT (SEQ ID NO. 32)
Amino Acid Sequence: GSGATNFSLLKQAGDVEENPGP (SEQ ID NO. 33)
CXCR3
Nucleotide Location: 6058-7161
Nucleotide Sequence:
ATGGTGCTGGAGGTGAGCGACCACCAGGTGCTGAACGACGCCGAGGTGGCCGCC
CTGCTGGAGAACTTCAGCAGCAGCTACGACTACGGCGAGAACGAGAGCGACAGC
TGCTGCACCAGCCCCCCCTGCCCCCAGGACTTCAGCCTGAACTTCGACCGCGCCT
TCCTGCCCGCCCTGTACAGCCTGCTGTTCCTGCTGGGCCTGCTGGGCAACGGCGC
CGTGGCCGCCGTGCTGCTGAGCCGCCGCACCGCCCTGAGCAGCACCGACACCTTC
CTGCTGCACCTGGCCGTGGCCGACACCCTGCTGGTGCTGACCCTGCCCCTGTGGG
CCGTGGACGCCGCCGTGCAGTGGGTGTTCGGCAGCGGCCTGTGCAAGGTGGCCG
GCGCCCTGTTCAACATCAACTTCTACGCCGGCGCCCTGCTGCTGGCCTGCATCAG
CTTCGACCGCTACCTGAACATCGTGCACGCCACCCAGCTGTACCGCCGCGGCCCC
CCCGCCCGCGTGACCCTGACCTGCCTGGCCGTGTGGGGCCTGTGCCTGCTGTTCG
CCCTGCCCGACTTCATCTTCCTGAGCGCCCACCACGACGAGCGCCTGAACGCCAC
CCACTGCCAGTACAACTTCCCCCAGGTGGGCCGCACCGCCCTGCGCGTGCTGCAG
CTGGTGGCCGGCTTCCTGCTGCCCCTGCTGGTGATGGCCTACTGCTACGCCCACA
TCCTGGCCGTGCTGCTGGTGAGCCGCGGCCAGCGCCGCCTGCGCGCCATGCGCCT
GGTGGTGGTGGTGGTGGTGGCCTTCGCCCTGTGCTGGACCCCCTACCACCTGGTG
GTGCTGGTGGACATCCTGATGGACCTGGGCGCCCTGGCCCGCAACTGCGGCCGC
GAGAGCCGCGTGGACGTGGCCAAGAGCGTGACCAGCGGCCTGGGCTACATGCAC
TGCTGCCTGAACCCCCTGCTGTACGCCTTCGTGGGCGTGAAGTTCCGCGAGCGCA
TGTGGATGCTGCTGCTGCGCCTGGGCTGCCCCAACCAGCGCGGCCTGCAGCGCCA
GCCCAGCAGCAGCCGCCGCGACAGCAGCTGGAGCGAGACCAGCGAGGCCAGCT
ACAGCGGCCTG (SEQ ID NO. 34)
Amino Acid Sequence:
MVLEVSDHQVLNDAEVAALLENFSSSYDYGENESDSCCTSPPCPQDFSLNFDRAFLP
ALYSLLFLLGLLGNGAVAAVLLSRRTALSSTDTFLLHLAVADTLLVLTLPLWAVDAA
VQWVFGSGLCKVAGALFNINFYAGALLLACISFDRYLNIVHATQLYRRGPPARVTLT
CLAVWGLCLLFALPDFIFLSAHHDERLNATHCQYNFPQVGRTALRVLQLVAGFLLPL
LVMAYCYAHILAVLLVSRGQRRLRAMRLVVVVVVAFALCWTPYHLVVLVDILMDL
GALARNCGRESRVDVAKSVTSGLGYMHCCLNPLLYAFVGVKFRERMWMLLLRLGC
PNQRGLQRQPSSSRRDSSWSETSEASYSGL (SEQ ID NO. 35)
GSG-P2A Site 6
Nucleotide Location: 7162-7227
Nucleotide Sequence:
GGCAGCGGCGCCACGAACTTCTCTCTGTTAAAGCAAGCAGGAGACGTGGAAGAA
AACCCCGGTCCT (SEQ ID NO. 36)
Amino Acid Sequence: GSGATNFSLLKQAGDVEENPGP (SEQ ID NO. 37)
Human Serum Albumin Signal Peptide 6
Nucleotide Location: 7228-7281
Nucleotide Sequence:
ATGAAGTGGGTGACCTTCATCAGCCTGCTGTTCCTGTTCAGCAGCGCCTACAGC
(SEQ ID NO. 38)
Amino Acid Sequence: MKWVTFISLLFLFSSAYS (SEQ ID NO. 39)
IL-21
Nucleotide Location: 7282-7680
Nucleotide Sequence:
CAGGGCCAGGACCGCCACATGATCAGAATGCGCCAGCTGATCGACATCGTGGAC
CAGCTGAAGAACTACGTGAACGACCTGGTGCCCGAGTTCCTGCCCGCCCCCGAG
GACGTGGAGACCAACTGCGAGTGGAGCGCCTTCAGCTGCTTCCAGAAGGCCCAG
CTGAAGAGCGCCAACACCGGCAACAACGAGCGCATCATCAACGTGAGCATCAAG
AAGCTGAAGCGCAAGCCCCCCAGCACCAACGCCGGCCGCCGCCAGAAGCACCGC
CTGACCTGCCCCAGCTGCGACAGCTACGAGAAGAAGCCCCCCAAGGAGTTCCTG
GAGAGATTCAAGAGCCTGCTGCAGAAGATGATCCACCAGCACCTGAGCAGCCGC
ACCCACGGCAGCGAGGACAGC (SEQ ID NO. 40)
Amino Acid Sequence:
QGQDRHMIRMRQLIDIVDQLKNYVNDLVPEFLPAPEDVETNCEWSAFSCFQKAQLK
SANTGNNERIINVSIKKLKRKPPSTNAGRRQKHRLTCPSCDSYEKKPPKEFLERFKSLL
QKMIHQHLSSRTHGSEDS (SEQ ID NO. 41)

The nucleic acid constructs disclosed herein encode polypeptides. We tend to use the term “protein” to refer to longer or larger amino acid polymers, and we tend to use the term “polypeptide” to refer to shorter sequences or to a chain of amino acid residues within a larger molecule (e.g., within a fusion protein) or complex. Both terms, however, are meant to describe an entity of two or more subunit amino acids, amino acid analogs, or other peptidomimetics, regardless of post-translational modification (e.g., amidation, phosphorylation or glycosylation). The subunits can be linked by peptide bonds or other bonds such as, for example, dicysteine, ester or ether bonds. The terms “amino acid” and “amino acid residue” refer to natural and/or unnatural or synthetic amino acids, which may be D- or L-form optical isomers. Full-length proteins, analogs, mutants, and fragments thereof are encompassed by this definition.

The amino acid sequence of the polypeptides disclosed herein can be identical to the wild-type sequences of appropriate components. Alternatively, any of the components can contain mutations such as deletions, additions, or substitutions. All that is required is that the variant polypeptide have at least 5% (e.g., 10%, 20%, 30%, 40%, 50%, 60%, 70%, 80%, 90%, 95%, 99%, 100%, or even more) of the ability of the polypeptide containing only wild-type sequences to specifically function. Substitutions will preferably be conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Variant polypeptides, e.g., those having one or more amino acid substitutions relative to a native polypeptide amino acid sequence, can be prepared and modified as described herein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine) Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Variant polypeptides having conservative and/or non-conservative substitutions can be screened for biological activity using suitable assays.

The component factors comprising the constructs disclosed here in can have the amino acid sequence of any of the sequences shown above. Thus the component factors can have the amino acid sequence of: SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31 SEQ ID NO. 33, SEQ ID NO. 35, SEQ ID NO. 37, SEQ ID NO. 39, or SEQ ID NO. 41. In some embodiments, the component factor can have an amino acid sequence that is at least about 70%, at least about 75%, at least about 80%, at least about 85%, at least about 90%, at least about 95%, at least about 96%, at least about 97%, at least about 98%, at least about 99% identical to the amino acid sequence of: SEQ ID NO. 3, SEQ ID NO. 5, SEQ ID NO. 7, SEQ ID NO. 9, SEQ ID NO. 11, SEQ ID NO. 13, SEQ ID NO. 15, SEQ ID NO. 17, SEQ ID NO. 19, SEQ ID NO. 21, SEQ ID NO. 23, SEQ ID NO. 25, SEQ ID NO. 27, SEQ ID NO. 29, SEQ ID NO. 31 SEQ ID NO. 33, SEQ ID NO. 35, SEQ ID NO. 37, SEQ ID NO. 39, or SEQ ID NO. 41.

An exemplary sequence for any of the component factors can be a reference sequence. For example, an MMP8 sequence can have the nucleic acid sequence of any of GenBank entries NM_001304441, NM_001304442, or NM_002424. An IL-7 sequence can have the nucleic acid sequence of any of GenBank entries NM_000880, NM_001199886, NM_001199887 or NM_001199888. A PD-1 sequence can include the extracellular domain of the nucleic acid sequence of GenBank entry NM_005018. A CXCR3 sequence can have the nucleic acid sequence of any of GenBank entries NM_001142797 or. NM_001504. An IL 21 sequence can have the nucleic acid sequence of any of GenBank entries NM_021803 or NM_001207006.

In some embodiments, this technology includes a nucleic acid encoding the above-listed factors, or a subordinate list of the above-listed factors.

In some embodiments, this technology includes the use of a lentivector, or other viral vector, encoding a list or subordinate list of such factors in CAR-based therapies.

As used herein, a viral vector is a DNA or RNA-based vector that is packaged by viral structural proteins into a viral particles. More specifically, the DNA or RNA contains sequences that are, typically, specifically recognized by one or more viral structural proteins that comprise the viral particle: upon DNA/RNA sequence-specific engagement by these proteins, the DNA or RNA carrying the genes of interest become encapsidated in a viral particle competent for subsequent infection in target cells.

As used herein, a lentivector is a special class of viral vectors derived from lentiviruses (e.g. HIV-1 or 2, or HTLV-1 or 2). They minimally contain the viral Long-Terminal Repeats (LTR) at the 5′ and 3′ end of the genome and a packaging signal (the w sequence); however, lentivectors that contain additional sequences, like the Rev-Response Element (RRE), and splicing sites may be result in more infectious, or higher titer progeny virus. As such, these sequences are included in the pMSlenti lentivector. A Self-Inactivating (SIN) LTR lentivector is a lentivector in which the 3′ LTR is truncated: more specifically, most of the LTR's U3 region, and all of the U5 region are deleted. During infection, these deletions are copied to the 5′ LTR during reverse transcription, resulting in a double-stranded DNA that is incapable of LTR-mediated transcription.

As used herein, a solid tumor is a neoplasm whose primary form is aggregated cellular masses constrained by a host-derived stroma; alternatively, aggregated cellular masses derived from metastases, and possible lacking a well-defined stroma. In contrast, most hematological malignancies consist of transformed immune cells that exist as single cells or small cellular aggregates.

As used herein, a Treg is a regulatory T cell whose primary function is to suppress CD4 T, CD8 T, NKT, and B cells that respond to endogenous, autologous antigens (autoantigens) derived from the host. In so doing, they express suppressive cytokines (e.g. IL-10, IL-35, and TGFβ) and surface ligands (e.g. CD39 and CD73) that render target T cells angergic, or result in target T cell depletion (e.g. perform and granzymes) [Reviewed in 16].

As described herein, a protease or proteinase is an enzyme whose primary function is the hydrolytic cleavage of proteins or a specific protein. In the case of Matrix Metalloproteinase 8 (MMP8) the targets are Collagens Type I, II, and III. Pro-MMP-8 is a precursor to mature MMP-8: autocatalytic cleavage of Pro-MMP-8 yields mature MMP-8.

As described herein, a cytokine is a diverse group of secreted proteins that induce cellular responses through binding to a cognate receptor. They include the interleukins, chemokines, interferons, and tumor necrosis factors. Chemokines, as described herein, are secreted proteins that drive cellular chemotaxis, or movement, in response to the chemokine binding a cognate chemokine receptor of the CCR, CXCR, CR, or CX3CR families. Fugetaxis is a subcategory of chemotaxis, denoting movement away from the stimulus (e.g. a chemokine).

As described herein, an immune cell is broad category of white blood cells (WBCs) of hematopoietic origin comprising the innate immune system (e.g. macrophages, monocytes, neutrophils, eosinophils, basophils, mast cells, dendritic cells, and NK cells) or the adaptive immune system (NKT cells, T cells, and B cells). However, in some instances “immune cell” includes cells of mesenchymal origin, such as follicular dendritic cells or stromal cells in the primary, secondary, or tertiary lymphoid tissues.

As described herein, a Bi-specific T cell Engager (BiTE) is a protein fusion of two single chain Fv's (scFv's): more specifically, it is canonical that one scFv targets CD3, and that the other targets a known antigen on cancer cells or autoreactive T cells; though, other iterations exist. As applies to this technology, the BiTE is a scFv fusion comprised of the humanized anti-CD3 antibody, OKT3, and the VAC69 scFv.

### EXAMPLES

### Example 1: VAC69 CAR Constructs

An exemplary VAC69 CAR construct is shown in FIG. 2. Encoded in this construct is a third-generation CAR fused to the VAC69 single-chain Fv (scFv) and the IgG1 heavy chain constant regions CH2 and CH3. Being a third generation CAR, the intracellular portion includes the transmembrane domain (TMD) and intracellular domain (ICD) of CD28, the ICD of CD3ζ, and the ICD of OX40: these are separated by glycine-glycine-glycine-serine (GGGS) linkers. Additionally, the other components are encoded downstream of an rdm3 Internal Ribosome Entry Site (IRES) and, when secreted, Gaussia Dura Luc signal peptide, which is denoted “Signal Peptide.” Other factors expressed include the following: Interleukin 7 (IL-7); a fusion of PD-1 extracellular domain (ECD) with 4-1BB TMD and ICD; Interleukin 21 (IL-21); Pro-matrix metalloproteinase 8 (Pro-MMP8); the chemokine receptor, CXCR3; dominant-negative Fas-Associated Death Domain (FADD); Interleukin 21 (IL-21); a Bi-specific T cell Engager (BiTE) composed of the VAC69 scFv and humanized OKT3 scFv in the domain architecture, VLVAC69-VHVAC69-VHOKT3-VLOKT3; and the puromycin resistance gene (PuroR) for selection of transduced cells. CXCR3 I162 was conservatively mutated to leucine (L) to eliminate a BamHI restriction endonuclease site. Similarly, same-sense mutations were introduced into the OKT3 VH and VL regions to eliminate XhoI sites.

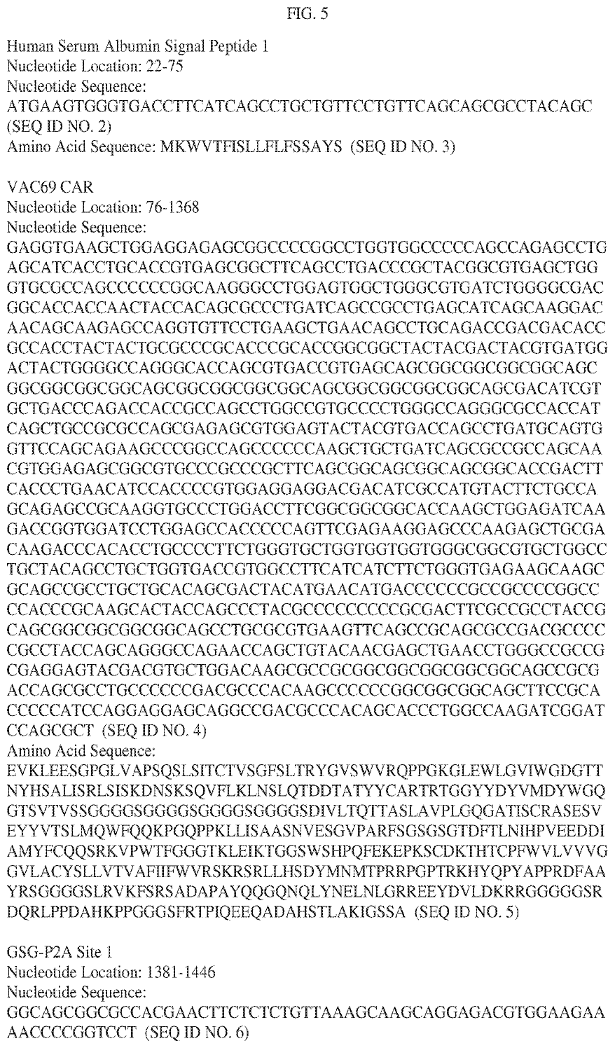

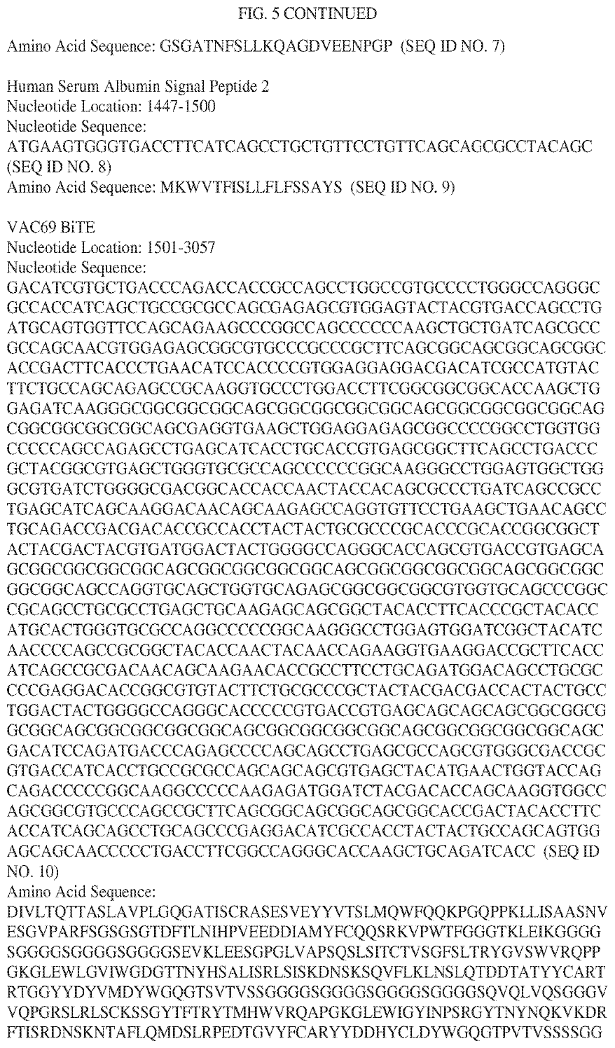

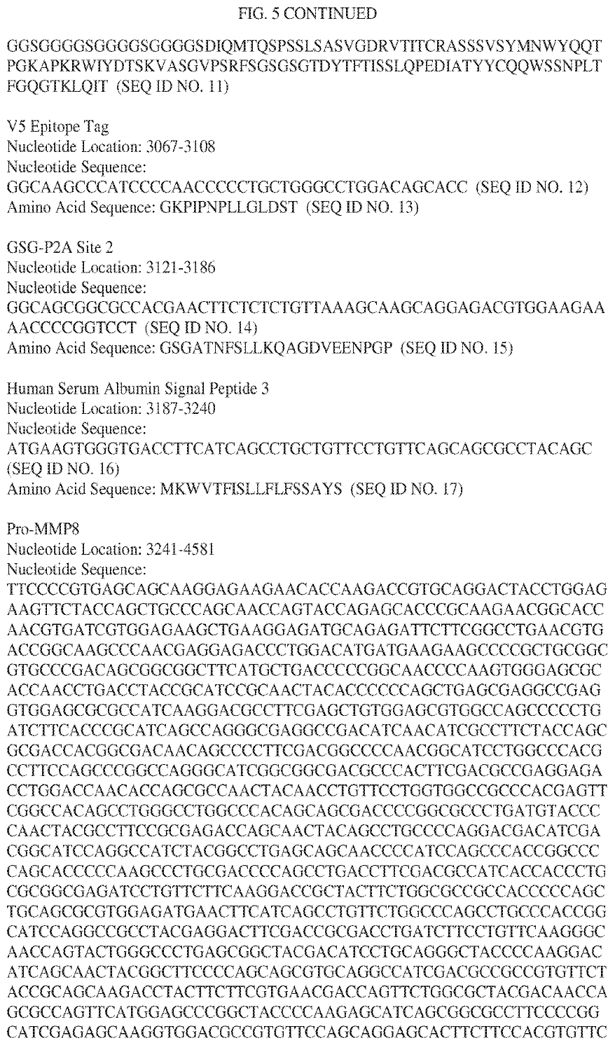

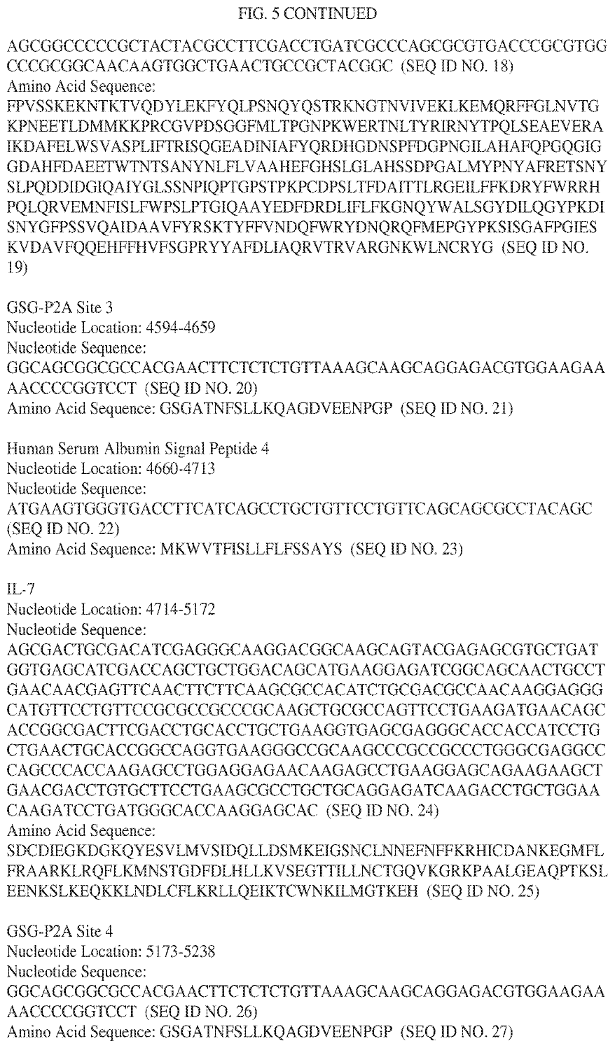

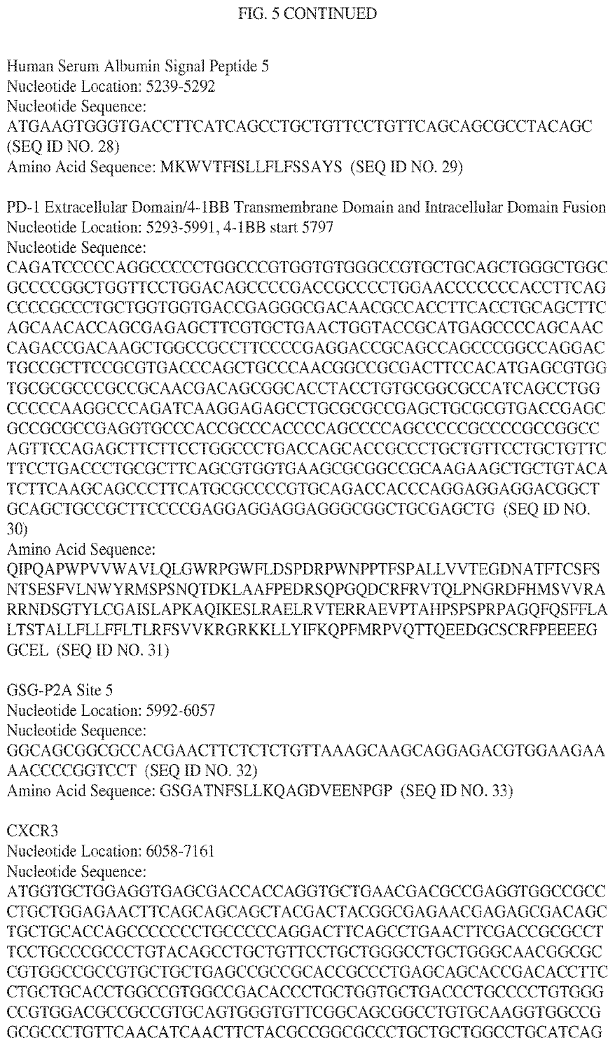

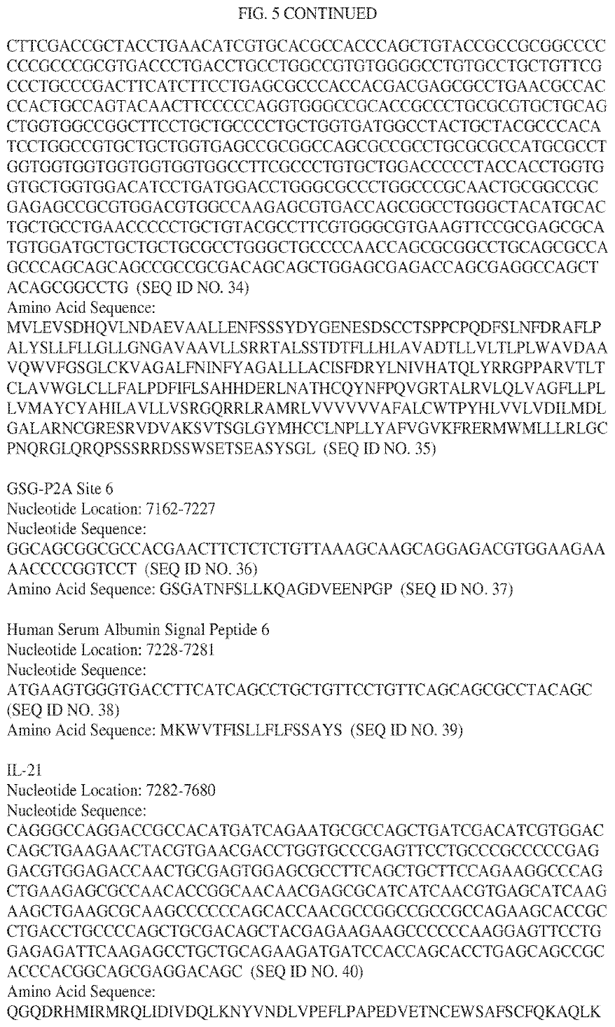

An alternative embodiment of the exemplary VAC69 CAR construct is shown in FIG. 3. The nucleotide sequence (SEQ ID NO. 1) of the alternative embodiment construct is shown in FIG. 4. This construct is reduced in size relative to the embodiment depicted in FIG. 2. In addition, the order of some of specific segments differs from the order shown in the embodiment in FIG. 2. The alternative embodiment construct does not include IRES, p60, and FADD segments. Unlike the embodiment shown in FIG. 2, the alternative embodiment construct includes P2A self cleaving peptide segments. The alternative embodiment construct includes the VAC69 single-chain Fv (scFv) and the IgG1 heavy chain constant regions CH2 and CH3, the transmembrane domain (TMD) and intracellular domain (ICD) of CD28, the ICD of CD3ζ, and the ICD of OX40, separated by glycine-glycine-glycine-serine (GGGS) linkers; GSG-P2A Site 1; human serum albumin signal peptide 2; VAC69 BiTE; V5 epitope tag; GSG-P2A Site 2; human serum albumin signal peptide 3; pro-MMP 8; GSG-P2A Site 3; human serum albumin signal peptide 4; IL-7; GSG-P2A Site 4; human serum albumin signal peptide 5; PD-1 Extracellular Domain/4-1BB Transmembrane Domain and Intracellular Domain Fusion; GSG-P2A Site 5; CXCR3; GSG-P2A Site 6; human serum albumin signal peptide 6; and IL 21. The location of the nucleotide sequences of the specific genes within SEQ ID NO. 1 is shown in FIG. 5. Corresponding amino acid sequences of these regions is also shown in FIG. 5.

### Example 2: Expression of VAC69 CAR Construct Encoded Factors

We analyzed the expression of factors encoded by the VAC69 CAR construct depicted in FIG. 3 in a cell-based assay system using the human embryonic kidney cell line, HEK293T. HEK293T cells were seeded at a density of 5×10⁵ cells per well in a 6-well plate and incubated overnight before PEI transfection, using 3 μg of VAC69 CAR vector DNA and 9 μg of PEI per well in a total volume of 300 μg of serum-free DMEM. Cells were incubated for 48 hours, without washing, at 37° C. and 5% CO₂ before harvesting.

For immunoblots, the supernatants were collected and the cells were lysed in the plate wells with 500 μg 1×LDS sample buffer (Thermo Fisher). Supernatants were admixed with 4×LDS sample buffer and 10× sample reducing agent (Thermo Fisher). All samples were heated at 70° C. for 10 minutes before loading onto Bis-Tris 4-12% SDS-PAGE gels (Thermo Fisher). Proteins were transferred using an iBlot device (Thermo Fisher) onto nitrocellulose membranes. Membranes were blocked with TBS-T containing 5% skim milk for 1 hour.

After blocking, membranes were stained overnight with 1 μg/mL of biotinylated anti-human IL-7 (Biolegend Catalog Number: 506601), anti-human MMP8 (Biolegend Catalog Number: 688002), a 1:5,000 dilution of HRP-conjugated anti-Strep Tag II antibody (Sigma Aldrich Catalog Number: 71591-3), or a 1:1,000 dilution of HRP-conjugated anti-V5 for BiTE staining (Biolegend Catalog Number: 680603). For MMP8, an anti-mouse IgG secondary HRP-conjugate (Thermo Fisher) was used at a dilution of 1:5,000. For IL-7, Pierce™ High Sensitivity Streptavidin-HRP was used for detection (Thermo Fisher Catalog Number: 21130).

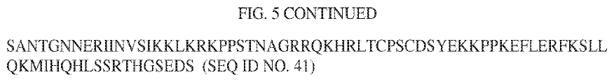

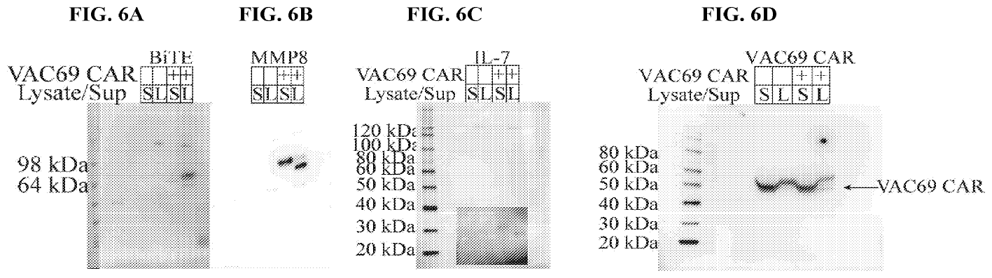

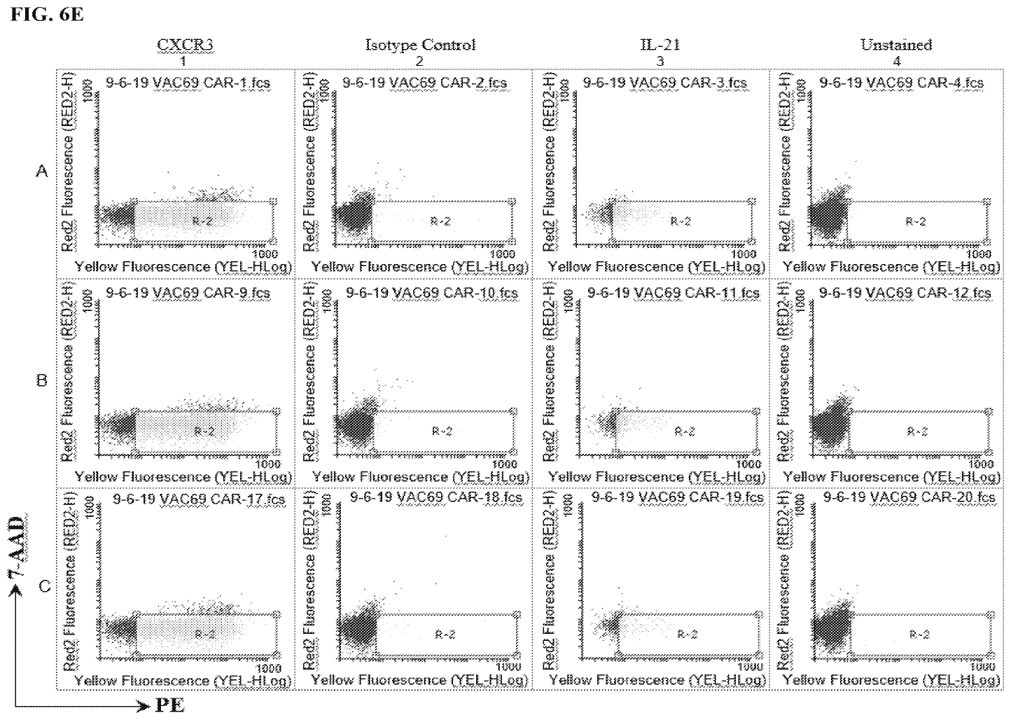

The results of the immunoblotting analyses are shown in FIG. 6A, FIG. 6B, FIG. 6C and FIG. 6D. We analyzed both cell supernatants and cell lysates from the transfected cells in order to monitor secretion of factors encoded by VAC69 CAR construct Each experiment also included cell supernatants and lysates from control cells that had not been transfected with the VAC69 CAR construct. In the experiment shown in FIG. 6A, supernatants and lysates from VAC69 CAR-transfected and untransfected cells were stained for anti-V5 epitope, which is tagged to the C-terminus of the VAC69 BiTE. As shown in FIG. 6A, the BiTE polypeptide was detected in cell lysates. The BiTE polypeptide was detected in at low levels in supernatants, indicating that the BiTE polypeptide was also secreted at low levels.

FIG. 6B is an immunoblot analyzing MMP8 polypeptide expression in both supernatants and lysates from VAC69 CAR-transfected and untransfected cells. As shown in FIG. 6B, MMP8 was detected in both supernatants and lysates, indicating that MMP 8 is both expressed in, and secreted from, VAC69 CAR-transfected cells.

FIG. 6C is an immunoblot analyzing IL-7 polypeptide expression in supernatants and lysates from VAC69 CAR-transfected and untransfected cells. As shown in FIG. 6C, IL-7 was detected in both supernatants and lysates, indicating that IL-7 is both expressed in, and secreted from, VAC69 CAR-transfected cells.

FIG. 6D is an immunoblot analyzing VAC69 CAR polypeptide expression in supernatants and lysates from VAC69 CAR-transfected and untransfected cells. As shown in FIG. 6D, a band corresponding to the expected size (50.9 kDa) of the VAC69 CAR was detected in the lysatesVAC from 69 CAR-transfected cells.

For flow cytometry, the cells were collected by trypsinization in 0.25% Trypsin-EDTA (Thermo Fisher) and then transferred to microtubes for staining. For IL-21 intracellular staining, cells were permeabilized and fixed using the True-Nuclear transcription factor buffer set as recommended by the manufacturer (Biolegend Catalog Number: 424401). Cells were stained with 5 μL of PE-conjugated anti-human CXCR3 (Biolegend Catalog Number: 353705), 5 μL of PE-conjugated anti-human IL-21 (Biolegend Catalog Number: 513003), or 5 μL of PE-conjugated mouse IgG1κ isotype control antibody (Biolegend Catalog Number: 400111) for 1 hour at 4° C. Cells were then washed twice with 1 mL of FACS Buffer (PBS, 2% BCS, 1 mM EDTA, and 0.1% sodium azide) before flow cytometric analysis.

The results of the flow cytometry analyses are shown in FIG. 6E. As shown in FIG. 6E, CXCR3 was robustly expressed on cell surfaces of HEK293T cells that had been transfected with the VAC69 CAR construct. IL 21 was moderately expressed intracellularly.

Taken together, the immunoblotting and the flow cytometry analyses show factors encoded by the VAC69 CAR construct are expressed at the polypeptide level and secreted accordingly from HEK293T cells that had been transfected with the VAC69 CAR construct.

### REFERENCES

1. Krueger P, Nitz C, Moore J, Foster R, Gelber O, Gelber C: Monoclonal antibody identifies a distinctive epitope expressed by human multiple myeloma cells. J Immunother Hagerstown Md. 1997 2001, 24:334-344.
2. Srivastava S, Riddell S R: Engineering CAR-T cells: Design concepts. Trends Immunol 2015, 36:494-502.
3. Sadelain M, Brentjens R, Riviere I: The basic principles of chimeric antigen receptor (CAR) design. Cancer Discov 2013, 3:388-398.
4. Kershaw M H, Westwood J A, Parker L L, Wang G, Eshhar Z, Mavroukakis S A, White D E, Wunderlich J R, Canevari S, Rogers-Freezer L, Chen C C, Yang J C, Rosenberg S A, Hwu P: A phase I study on adoptive immunotherapy using gene-modified T cells for ovarian cancer. Clin Cancer Res Off J Am Assoc Cancer Res 2006, 12(20 Pt 1):6106-6115.
5. Kakarla S, Gottschalk S: CAR T cells for solid tumors: armed and ready to go?. Cancer J Sudbury Mass 2014, 20:151-155.
6. Li H, Fan X, Houghton J: Tumor microenvironment: the role of the tumor stroma in cancer. J Cell Biochem 2007, 101:805-815.
7. Kessenbrock K, Plaks V, Werb Z: Matrix Metalloproteinases: Regulators of the Tumor Microenvironment. Cell 2010, 141:52-67.
8. Gutierrez-Fernandez A, Fueyo A, Folgueras A R, Garabaya C, Pennington C J, Pilgrim S, Edwards D R, Holliday D L, Jones J L, Span P N, Sweep FCGJ, Puente X S, López-Otín C: Matrix metalloproteinase-8 functions as a metastasis suppressor through modulation of tumor cell adhesion and invasion. Cancer Res 2008, 68:2755-2763.
9. Rocks N, Paulissen G, Quesada-Calvo F, Munaut C, Gonzalez M-LA, Gueders M, Hacha J, Gilles C, Foidart J-M, Noel A, Cataldo D D: ADAMTS-1 metalloproteinase promotes tumor development through the induction of a stromal reaction in vivo. Cancer Res 2008, 68:9541-9550.
10. Tang X, Li Q, Zhu Y, Zheng D, Dai J, Ni W, Wei J, Xue Y, Chen K, Hou W, Zhang C, Feng X, Liang Y: The advantages of PD1 activating chimeric receptor (PD1-ACR) engineered lymphocytes for PDL1+ cancer therapy. Am J Transl Res 2015, 7:460-473.
11. Markley J C, Sadelain M: IL-7 and IL-21 are superior to IL-2 and IL-15 in promoting human T cell-mediated rejection of systemic lymphoma in immunodeficient mice. Blood 2010, 115:3508-3519.
12. Gajewski T F, Schreiber H, Fu Y-X: Innate and adaptive immune cells in the tumor microenvironment. Nat Immunol 2013, 14:1014-1022.
13. Allison J, Thomas H E, Catterall T, Kay T W H, Strasser A: Transgenic expression of dominant-negative Fas-associated death domain protein in beta cells protects against Fas ligand-induced apoptosis and reduces spontaneous diabetes in nonobese diabetic mice. J Immunol Baltim Md. 1950 2005, 175:293-301.
14. Casares N, Rudilla F, Arribillaga L, Llopiz D, Riezu-Boj J I, Lozano T, López-Sagaseta J, Guembe L, Sarobe P, Prieto J, Borrás-Cuesta F, Lasarte J J: A peptide inhibitor of FOXP3 impairs regulatory T cell activity and improves vaccine efficacy in mice. J Immunol Baltim Md. 1950 2010, 185:5150-5159.
15. Dao T, Pankov D, Scott A, Korontsvit T, Zakhaleva V, Xu Y, Xiang J, Yan S, de Morals Guerreiro M D, Veomett N, Dubrovsky L, Curcio M, Doubrovina E, Ponomarev V, Liu C, O'Reilly R J, Scheinberg D A: Therapeutic bispecific T-cell engager antibody targeting the intracellular oncoprotein WT1. Nat Biotechnol 2015, 33:1079-1086.
16. Schmidt A, Oberle N, Krammer P H: Molecular Mechanisms of Treg-Mediated T Cell Suppression. Front Immunol 2012, 3.

What is claimed is:

1. A construct comprising a nucleic acid sequence encoding:
a) a chimeric antigen receptor (CAR) comprising a single chain variable fragment from monoclonal antibody VAC69;
b) one or more cytokines; and
c) one or more of:
i) a matrix metaloproteinase;
ii) a PD1 fusion protein;
iii) a chemokine receptor;
iv) a dominant negative or nonfunctional immunosuppressive or toxic receptor.
v) a FOXP3 inhibitory peptide P60;
vi) a Bi-specific T Cell Engager (BiTE).

2. The construct of claim 1, wherein the single chain variable fragment from monoclonal antibody VAC69 is humanized.

3. The construct of claim 1, wherein the CAR is a third-generation CAR.

4. The construct of claim 1, wherein the CAR comprises the transmembrane and intracellular domains of CD28, the intracellular domain of CD3ζ, and the intracellular domain of OX40.

5. The construct of claim 1, wherein the CAR further comprises a glycine-glycine-glycine-serine (GGGS) linker.

6. The construct of claim 1, wherein the CAR comprises a nucleotide sequence at least 90% identical to SEQ ID NO. 4.

7. The construct of claim 1, wherein the CAR comprises the nucleotide sequence of SEQ ID NO. 4.

8. The construct of claim 1, wherein the PD1 fusion protein comprises a PD-1 extracellular domain (ECD) and a transmembrane domain (TMD) and an intracellular domain (ICD) of 4-1BB.

9. The construct of claim 1, wherein the PD1 fusion protein comprises a nucleotide sequence at least 90% identical to SEQ ID NO. 30.

10. The construct of claim 1, wherein the PD1 fusion protein comprises the nucleotide sequence of SEQ ID NO. 30.

11. The construct of claim 1, wherein the cytokine is selected from the group consisting of IL-7 and IL 21.

12. The construct of claim 1, wherein the cytokine is IL-7, IL-21, or both IL-7 and IL 21.

13. The construct of claim 11, wherein the IL-7 comprises a nucleotide sequence at least 90% identical to SEQ ID NO. 24.

14. The construct of claim 11, wherein the IL-7 comprises the nucleotide sequence of SED NO. 24.

15. The construct of claim 11, wherein the IL-21 comprises a nucleotide sequence at least 90% identical to SEQ ID NO. 40.

16. The construct of claim 11, wherein the IL-21 comprises the nucleotide sequence of SED NO. 40.

17. The construct of claim 1, wherein the matrix metaloproteinase is Pro-MMP-8.

18. The construct of claim 17, wherein the Pro-MMP-8 comprises a nucleotide sequence at least 90% identical to SEQ ID NO. 18.

19. The construct of claim 17, wherein the Pro-MMP-8 comprises the nucleotide sequence of SEQ ID NO. 18.

20. The construct of claim 1, wherein the dominant negative or nonfunctional immunosuppressive or toxic receptor is a dominant negative Fas-associated death domain protein (FADD).

21. The construct of claim 1, where in the Bi-specific T Cell Engager (BiTE) comprises a fusion protein comprising a single chain variable fragment from monoclonal antibody VAC69 and a humanized single chain variable fragment monoclonal antibody OKT3.

22. The construct of claim 1, where in the Bi-specific T Cell Engager (BiTE) comprises comprises a nucleotide sequence at least 90% identical to SEQ ID NO. 10.

23. The construct of claim 1, where in the Bi-specific T Cell Engager (BiTE) comprises comprises the nucleotide sequence of SEQ ID NO. 10.

24. The construct of claim 1, wherein the construct further comprises one or more of a signal peptide, a self cleaving peptide, an epitope tag, an internal ribosome entry site (RES), or a selectable marker.

25. The construct of claim 24, wherein the signal peptide is a human serum albumin signal peptide.

26. The construct of claim 25, wherein the human serum albumin signal peptide comprises a nucleotide sequence at least 90% identical to SEQ ID NO. 2, 8, 16, 22, 28, or 38.

27. The construct of claim 25, wherein the human serum albumin signal peptide comprises comprises the nucleotide sequence of SEQ ID NO. 2, 8, 16, 22, 28, or 38.

28. The construct of claim 24, wherein self-cleaving peptide is a P2A peptide.

29. The construct of claim 28, wherein the P2A peptide comprises a nucleotide sequence at least 90% identical to SEQ ID NO. 6, 14, 20, 26, 32, or 36.

30. The construct of claim 28 wherein the P2A peptide comprises the nucleotide sequence of SEQ ID NO. 6, 14, 20, 26, 32, or 36.

31. The construct of claim 24, wherein the epitope tag comprises a V5 epitope tag.

32. The construct of claim 31, wherein the V5 epitope tag comprises a nucleotide sequence at least 90% identical to SEQ ID NO. 12.

33. The construct of claim 31, wherein the V5 epitope tag comprises the nucleotide sequence of SEQ ID NO. 12.

34. A construct comprising a nucleic acid sequence encoding:
a) a chimeric antigen receptor (CAR) comprising a single chain variable fragment from monoclonal antibody VAC69;
b) a Bi-specific T Cell Engager (BiTE) comprising a fusion protein comprising a single chain variable fragment from monoclonal antibody VAC69 and a humanized single chain variable fragment monoclonal antibody OKT3;
c) Pro-MMP-8
d) IL-7;
e) a fusion protein comprising a PD1-extracellular domain and a 4-1BB transmembrane and intracellular domain;
f) a CXCR3 chemokine receptor; and
g) IL-21.

35. The construct of claim 34, further comprising one or more of a signal peptide, a self cleaving peptide, and an epitope tag.

36. The construct of claim 34, wherein the construct comprises a nucleotide sequence at least 90% identical to SEQ ID NO. 1.

37. The construct of claim 34, wherein the construct comprises the nucleotide sequence of SEQ ID NO. 1.

38. A vector comprising the construct of any of claims 1-37.

39. The vector of claim 38, wherein the vector is a lentiviral vector.

40. The vector of claim 39, wherein the lentiviral vector is a self inactivating lentiviral vector.

41. A host cell comprising the vector of any one of claims 38-40.

42. A pharmaceutical composition comprising the construct of any one of claims 1-37.

43. A method of treating a malignancy, the method comprising administering a pharmaceutical composition comprising the construct of any one of claims 1-37, the vector of any one of claims 38-40, or the host cell of claim 41 to a subject having a malignancy.

44. The method of claim 43, wherein the malignancy is a solid tumor.
